(19) United States
(12) Patent Application Publication
(10) Pub. No.: US 2015/0374610 A1
(43) Pub. Date: Dec. 31, 2015

(54) TOPICAL PROTECTIVE POLYMERIZED NANOPARTICLES IN AN ACTIVE OR BIOACTIVE ARRAY, METHODS FOR PREPARING SAME AND USES THEREOF

(71) Applicants: CENTRE NATIONAL DE LA RECHERCHE SCIENTIFIQUE, Paris (FR); UNIVERSITÉ NICE SOPHIA ANTIPOLIS, Nice (FR)

(72) Inventors: Frédéric Guittard, Nice (FR); Sonia Amigoni, Nice (FR); Elisabeth Taffin de Givenchy, Nice (FR); Arnaud Zenerino, Blausasc (FR); Denis Josse, Vaulnaveys Le Haut (FR)

(73) Assignees: CENTRE NATIONAL DE LA RECHERCHE SCIENTIFIQUE, Paris (FR); UNIVERSITE NICE SOPHIA ANTIPOLIS, Nice (FR)

(21) Appl. No.: 14/769,327

(22) PCT Filed: Feb. 21, 2014
(86) PCT No.: PCT/FR2014/000043

§ 371 Date: Aug. 20, 2015

(30) Foreign Application Priority Data
Feb. 21, 2013 (FR) 13/00396

(51) Int. Cl. A61K 8/81; A61Q 17/00; C08F 8/42; A61K 8/02

(52) U.S. Cl. CPC A61K 8/8152; A61K 8/0241; A61Q 17/00; C08F 8/42; A61K 2800/651; A61K 2800/75; A61K 2800/614

(57) ABSTRACT

The invention concerns a compound formed by functionalised micro- or nanoparticles covalently associated with rheology-modifying polymers. The invention is characterised in that the functionalised micro- or nanoparticles are functionalised inorganic or metal oxide micro- or nanoparticles, in particular aluminium oxide (Al₂O₃), copper oxide (CuO), iron oxide (Fe₃O₄ or γ-Fe₂O₃), magnesium oxide (MgO), silica (SiO₂), titanium dioxide (TiO₂) or zinc oxide (ZnO), having a nominal diameter of between 1 and 1500 nm; and in that the rheology-modifying or -adapting polymers are chosen from non-associative polymers or associative polymers. The invention applies to protection or decontamination of the skin.

The present invention relates to hybrid organic/inorganic compounds formed by functionalized micro- or nanoparticles of aluminum oxide (Al₂O₂), copper oxide (CuO), iron oxide (Fe₃O₄ or γ-Fe₂O₃), magnesium oxide (MgO), silica (SiO₂), titanium dioxide (TiO₂) or zinc oxide (ZnO), grafted covalently onto rheology-modifying polymers. More particularly, the invention relates to amine functionalized silica or titanium dioxide micro- or nanoparticles, grafted onto polymers to form an active or bioactive array. The invention further relates to protective topical agents comprising same, the synthesis methods thereof and the uses thereof particularly for protection or decontamination of the skin.

Contaminations with biological or chemical hazard agents may be multiple. Biological hazard agents are generally bacteria, viruses or toxins. Chemical hazard agents are generally organophosphate neurotoxic compounds, or vesicants.

Since the appearance of chemical terrorism and bioterrorism, numerous reflections to protect humans have been initiated. In interventions, for example after a terrorist act or in the case of use of toxins by soldiers, wearing protective clothing is mandatory. This type of clothing should also be used in the agricultural sector and in some industries. Indeed, the use for example of hazardous pesticides may contaminate the body and cause significant lesions or even the death of the user. The major problem of protective clothing is that it gives rise to changes in the operational capabilities of the intervention team and a reduction in their sensory capabilities (vision, hearing, manual dexterity, etc.) Furthermore, it rapidly involves restrictions of use as it may be poorly fitted or torn and thus expose the skin to toxic substances.

For all of the above reasons, it is necessary to find novel protection means restoring all or part of the users' capabilities. Before defining these protection means, it is important to identify the hazard agents, along with the properties thereof and the skin penetration mode thereof.

Toxic chemicals may be classified according to a plurality of criteria such as the volatility thereof, the military use thereof or the hemotoxic, vesicant, suffocating, neurotoxic, incapacitating, neutralizing effects thereof.

At the present time, there is primarily an interest in protection against vesicant agents and organophosphate compounds which represent the main chemical threat. Vesicant agents are essentially represented by sulfur or nitrogen yperites and more moderately by lewisite and phosgene oxime. Organophosphate compounds (OPCs) include pesticides (OPPs) and organophosphate neurotoxins (OPNs). OPPs are generally phosphates or phosphorothioates, containing O,O-dimethyl or O,O-diethyl substituents on the phosphorus atom. They are generally indirect cholinesterase inhibitors, i.e. they only become active after metabolic transformation: S-oxidation of the P═S bond. This is conveyed by a considerable latency between exposure and the onset of signs of intoxication. Organophosphate pesticides have replaced organochlorine compounds which were highly remanent despite the higher toxicity of OPPs. The first compounds such as parathion (O,O-diethyl and O-p-nitrophenyl phosphorothioate), malathion (S-(1,2-dicarbethoxyethyl) and O,O-dimethyl di-thiophosphate) and paraoxon (diethyl p-nitrophenyl phosphate) are powerful cholinesterase inhibitors. Organophosphate pesticides are extremely toxic and induce severe intoxication particularly in the agricultural sector. The World Health Organization (WHO) has estimated that, each year, 1 million cases of severe pesticide poisoning occur, with some 220,000 deaths. The risk of intoxication by pesticides is high due to frequent contact when spraying pesticides from ground level or the air and handling.

The first organophosphate neurotoxins (OPNs) synthesized were G agents. They notably include GA or Tabun agent, GB or Sarin agent and GD or Soman agent. They are fluorophosphonic or phosphoramidic acid derivative esters. Other organophosphate neurotoxins (OPNs) are V agents.

Exposure to excessive concentrations of such agents may induce intense bronchial, salivary, ocular and intestinal secretion, followed by sweating, bradycardia, muscle contractions, tremors, paralysis, loss of consciousness, convulsions, respiratory system dysfunction and death.

As such, the best way to prevent percutaneous toxicity of chemical agents is to never allow them to come into contact with the skin.

As mentioned above, prevention of skin contamination essentially involves wearing protections such as coveralls, masks or gloves. However, areas of exposure to these agents may remain, during movements of the human subject or due to lack of protection, for example if the protective equipment is poorly fitted or unsuitable. Moreover, transfer of contamination to the skin is also possible during the undressing phase.

There is thus a need to develop further protection means which are well tolerated by the user thereof, are easy to use, resistant to the external environment and which do not impede the wearer.

For this, the use of protective topical agents makes it possible to provide alternative or additional protection against irritants and aggressive substances from the environment, such as for example microorganisms, chemicals, vesicant agents and organophosphate compounds.

The majority of products sold under the term “protective topical agents” (PTs) includes both merely emollient cosmetic products and formulations potentially performing a barrier function against aggressive chemicals. These topical agents are intended to be used in at-risk environments. Protective topical agents are used in numerous fields such as industry or personal use. They can thus:
prevent skin contact allergy of numerous metals including nickel, cobalt, chromium, palladium and gold;
be used as solar filters for filtering UVA and UVB based on titanium dioxide nanoparticles or silicone emulsion;
be used as insect-repellants;
be used for chemical protection against acids, bases, surfactants and organic solvents.

Protective topical agents based on perfluorinated compounds are good means of protecting the body and allow a certain ease of use. Such topical agents are notably described in the document Zhang J.; Smith E. W.; Surber C.; Galenical principles in skin protection, Curr. Probl. Dermatol. 2007, 34, 11-18; and in the documents U.S. Pat. No. 5,607,979 and GB 2,314,020.

However, such topical agents do not break down the contaminant agents and the protective effect thereof is limited notably in cases of exposure to chemical fumes.

A second generation of protective topical agents consisting of incorporating organic or inorganic agents, suitable for neutralizing chemical contaminants has also been developed. Such topical agents are notably described in the document Koper O.; Lucas E.; Klabunde K. J.; Development of reactive topical skin protectants against sulfur mustard and nerve agents, J. Appl. Toxicol., 1999, 19, 59-70; or in the document Saxena A.; Srivastava A. K.; Singh B.; Goyal A.; Removal of sulphur mustard, sarin and stimulants on impregnated silica nanoparticles, J. Hazard. Mater., 2012, 211-212, 226-232).

The protective efficacy thereof is partially established and not optimized notably due to agglomeration of the micro- and/or nanoparticulate agents.

There is thus currently a need to develop novel protective topical agents which are more effective, have little or no toxicity, are easy to implement and which have a broad spectrum of action against biological and/or chemical hazard agents. Preferably, such protective topical agents are suitable for destroying the biological and/or chemical hazard agents. As such, these agents do not penetrate the subject's skin.

Furthermore, such topical agents should ideally be suitable for use by means of simple, quick and inexpensive methods and should have a homogeneous formulation.

In this context, the Applicant has developed a novel concept consisting of grafting micro- or nanoparticles, known for the neutralizing effects thereof in respect of toxic chemical and/or biological agents, with polymers, notably rheology modifiers, promoting the integration and homogeneous dispersion of the micro- or nanoparticles in a topical formulation but also by preventing the release of the micro- or nanoparticles.

The solution to the stated problem thus relates to a compound formed by functionalized micro- or nanoparticles, covalently associated with rheology-modifying polymers, characterized in that:
the functionalized micro- or nanoparticles are functionalized inorganic or metal oxide micro- or nanoparticles, in particular aluminum oxide (Al₂O₂), copper oxide (CuO), iron oxide (Fe₃O₄ or γ-Fe₂O₃), magnesium oxide (MgO), silica (SiO₂), titanium dioxide (TiO₂) or zinc oxide (ZnO), having a nominal diameter of between 1 and 1500 nm;
the rheology-modifying or -adapting polymers are chosen from non-associative polymers or associative polymers.

Surprisingly, the Applicant notably succeeded in demonstrating, as illustrated in example 11, that the compounds according to the invention are suitable for obtaining excellent protective topical agents limiting the transmembrane penetration of toxic agents, such as paraoxon. The Applicant also succeeded in demonstrating, using ecotoxicity tests featured in example 12, that the compounds according to the invention have a low environmental toxicity.

The present invention also secondly relates to a protective topical agent comprising a compound according to the invention, in a pharmaceutically and/or cosmetically acceptable medium.

It also thirdly relates to a compound or a protective topical agent according to the invention used as a medicinal product.

It fourthly relates to a compound or a protective topical agent according to the invention, for use in the prevention of skin irritations or allergies.

It fifthly relates to the use of a compound or a protective topical agent according to the invention, for protection or decontamination of the skin, notably due to biological and/or chemical hazard agents.

It sixthly relates to a method for synthesizing a compound according to the invention comprising steps for:
mixing a coupling agent with a catalyst;
adding the mixture obtained to a solution of rheology-modifying or adapting polymers chosen from non-associative or associative polymers, in water;
stirring the reaction mixture;
adding functionalized micro- or nanoparticles of aluminum oxide (Al₂O₂), magnesium oxide (MgO), silica (SiO₂), titanium dioxide (TiO₂), zinc oxide (ZnO) or copper oxide (CuO) having a nominal diameter between 5 and 1500 nm, previously dispersed in aqueous phase in the reaction mixture;
purifying the reaction mixture by dialysis; and
retrieving the compound formed by one or a plurality of amine functionalized micro- or nanoparticles covalently associated with one or a plurality of rheology-modifying polymers.

It seventhly relates to methods for synthesizing compounds according to the invention.

Finally, it lastly relates to a rheology-modifying or adapting polymer chosen from non-associative or associative polymers suitable for use in the compounds according to the invention.

The various compounds described above may notably be synthesized according to the various methods, the main steps whereof are described in examples 1 to 7.

The invention will be understood more clearly on reading the following non-limiting description, written with reference to the appended figures, wherein:

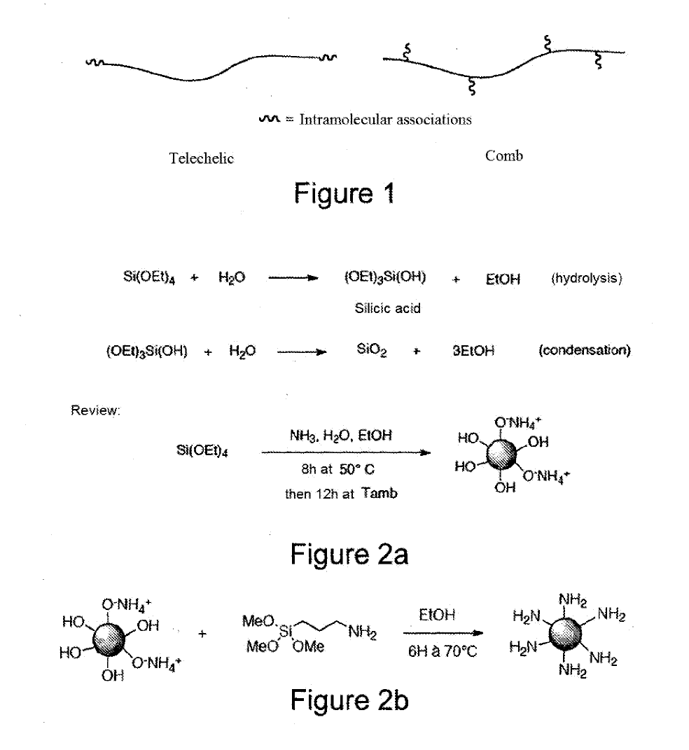

FIG. 1 is a schematic representation of telechelic and comb type compounds;

FIG. 2a represents synthesis of micro- or nanoparticles of silica using the Stöber method;

FIG. 2b illustrates functionalization of micro- or nanoparticles of silica with AMS (3-aminopropyl)trimethoxysilane);

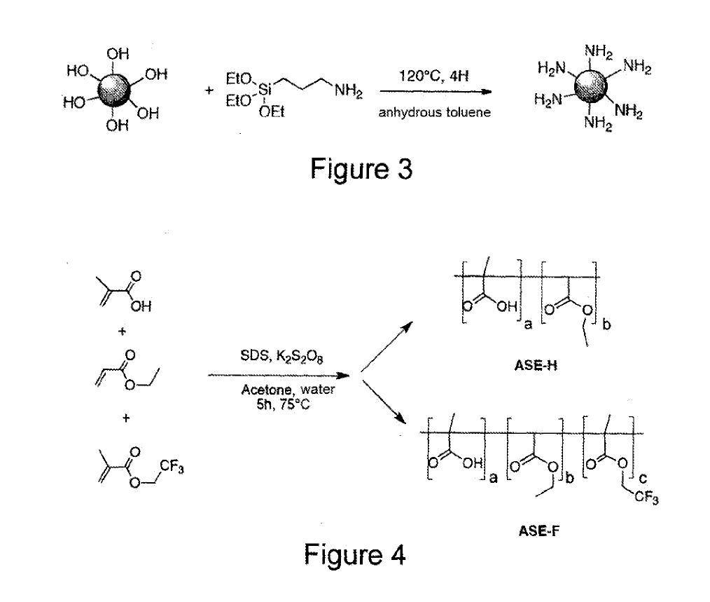

FIG. 3 represents functionalization of commercial nanoparticles with AMS;

FIG. 4 is a schematic illustration of synthesis of ASE-H and ASE-F non-associative polymers;

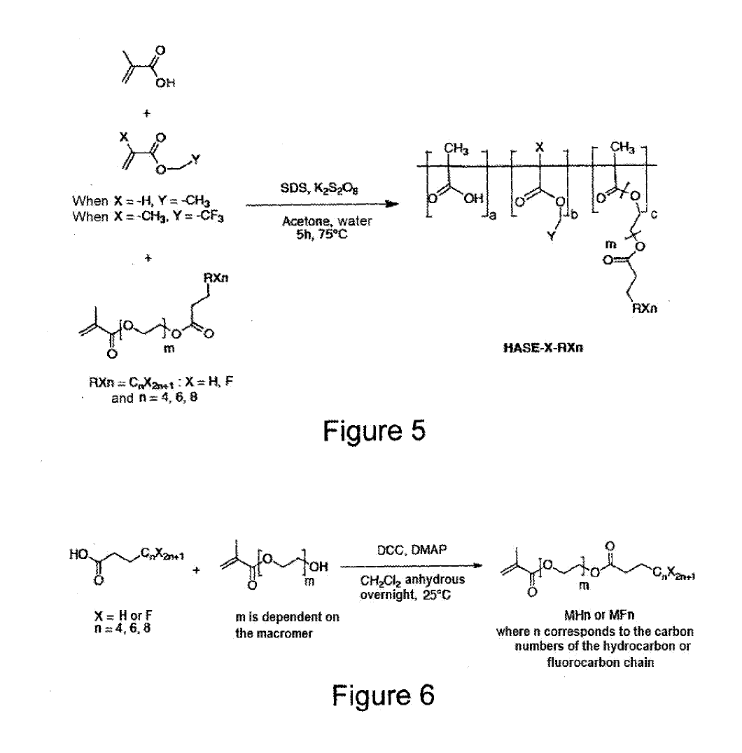

FIG. 5 is a schematic illustration of synthesis of HASE associative polymers;

FIG. 6 is a schematic representation of synthesis of macromers intended to be integrated in HASE associative polymers;

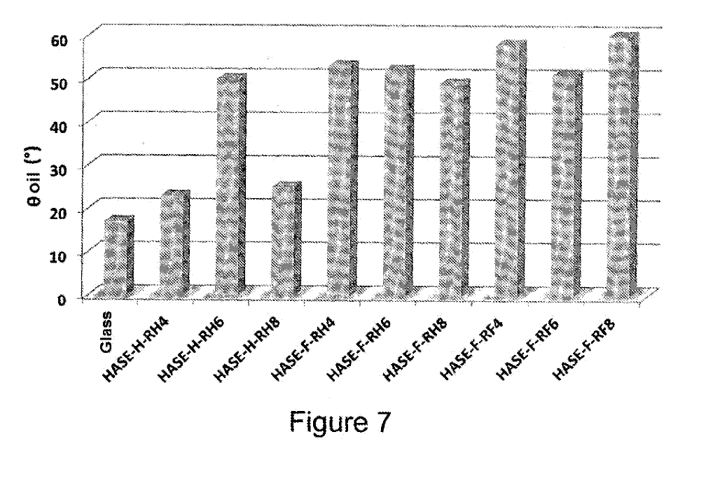

FIG. 7 represents studies of the contact angles of olive oil on various polymers previously deposited on a glass plate;

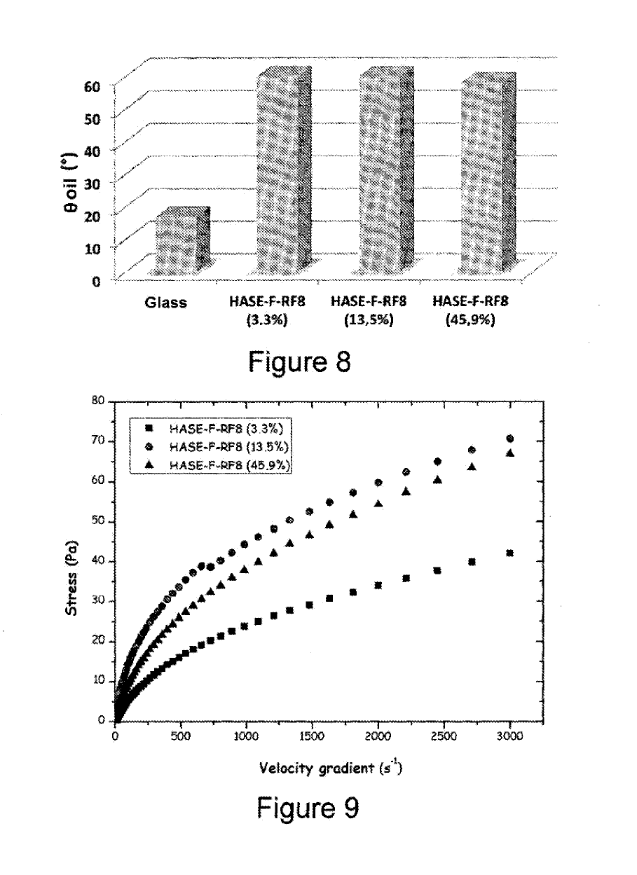

FIG. 8 represents studies of the contact angles of olive oil for various HASE-F-RF8 polymers containing 3.3; 13.5 and 45.9% mol. macromers;

FIG. 9 represents a flow analysis for the polymers HASE-F-RF8 (3.3% mol. macromers), (13.5% mol. macromers) and (45.9% mol. macromers).

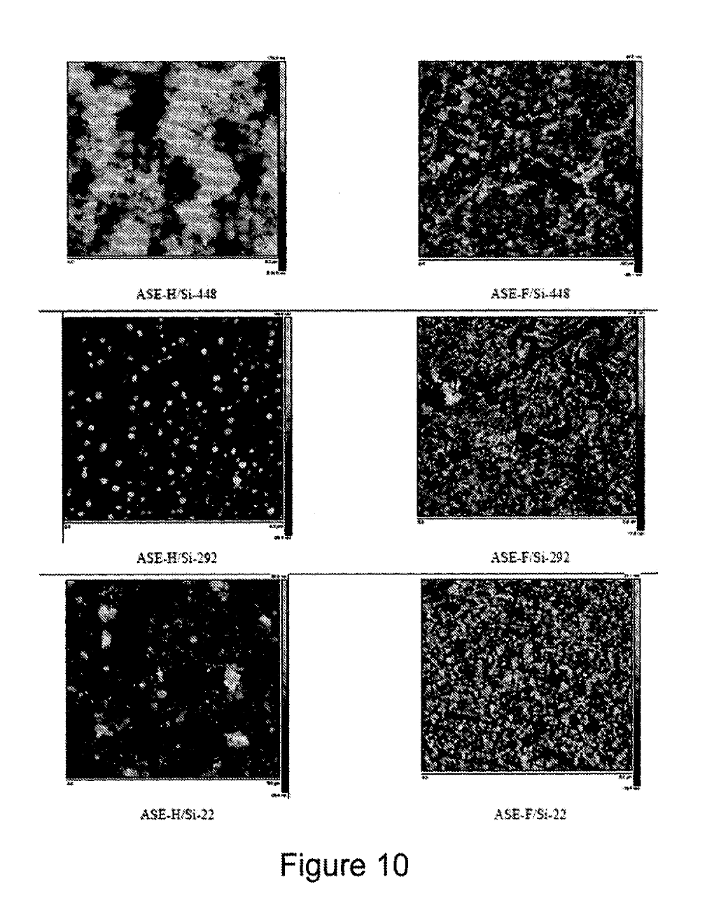

FIG. 10 represents images of the film topographies in neutral medium of the polymer/silica compounds according to the invention (at 1 equivalent);

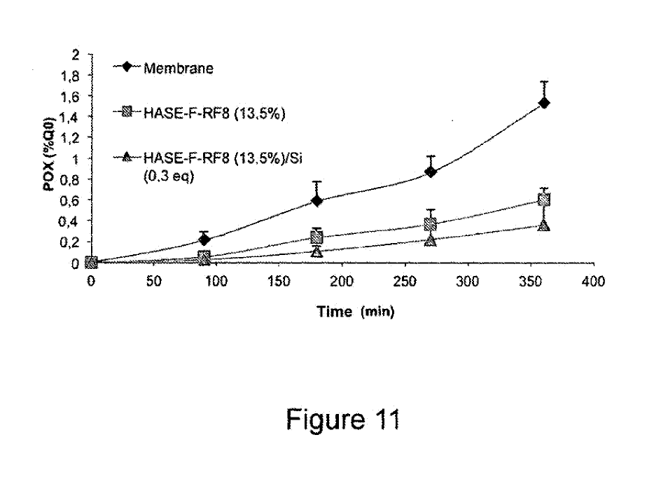

FIG. 11 represents an evaluation of topical agents (HASE-F-RF8 (13.5% mol. macromers)/Si and HASE-F-RF8 (13.5% mol. macromers)) with respect to transmembrane penetration of paraoxon;

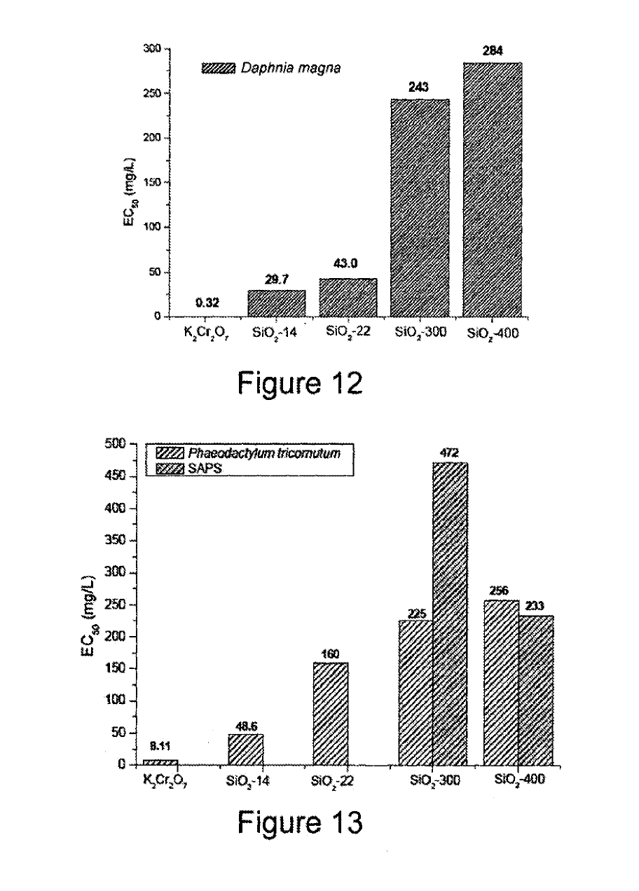

FIG. 12 presents the measurements of the EC₅₀ for Daphnia magna of water suspensions of nanoparticles of pure silica (SiO2-14) and functionalized silica (SiO2-22; -300 and -400);

FIG. 13 represents measurements of the EC₅₀ of Phaeodactylym tricornutum and Chlorella vulgaris (SAPS) of water suspensions of nanoparticles of pure silica (SiO2-14) and functionalized silica (SiO2-22; -300 and -400);

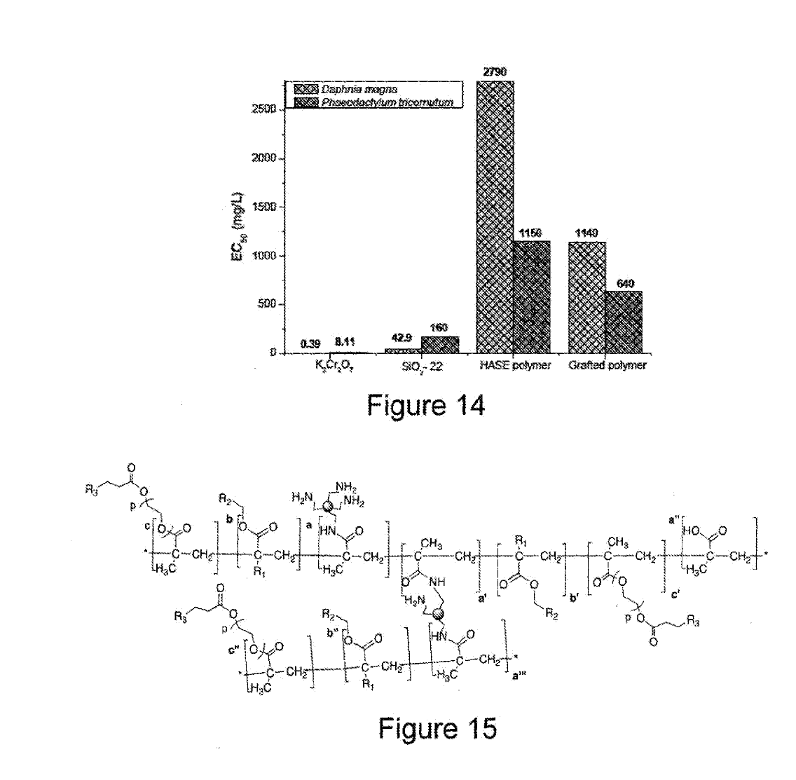

FIG. 14 represents measurements of the EC₅₀ of Daphnia magna and Phaeodactylym tricornutum of water suspensions of the polymer HASE-H-RH8 (1.3% mol. macromers) (“HASE polymer”) and the compound HASE-H-RH8 (1.3% mol. macromer)/Si (0.3 eq) (“Grafted Polymer”).

FIG. 15 represents a possible general structure of compounds according to the invention suitable for obtaining new-technology protection;

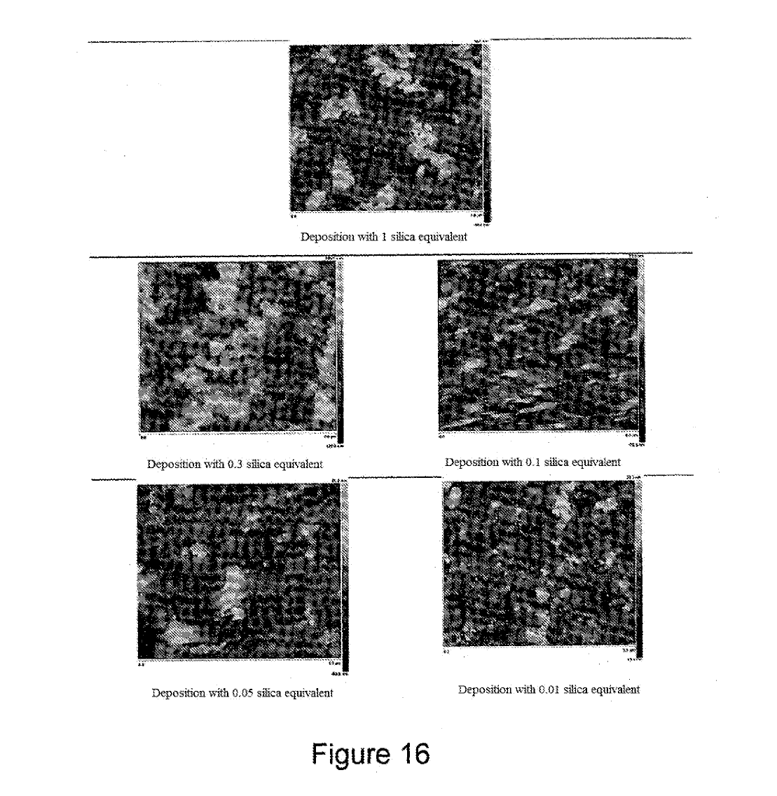

FIG. 16 represents images of the film topographies in neutral medium of the compounds HASE-F-RF8 (3.3%)/Si-22 with 1, 0.3; 0.1; 0.05 and 0.01 equivalent; and

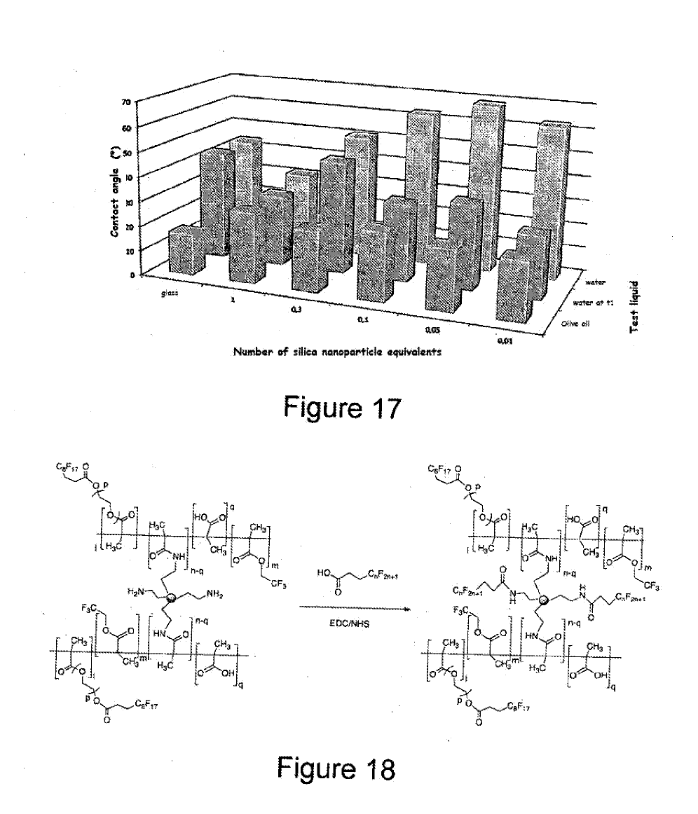

FIG. 17 illustrates a histogram containing the contact angle values for water, water after 1 minute (water at t1) and olive oil according to the number of silica equivalent contain in the various polymer/silica depositions;

FIG. 18 is a schematic illustration of synthesis of an organic/inorganic polymer array according to the invention;

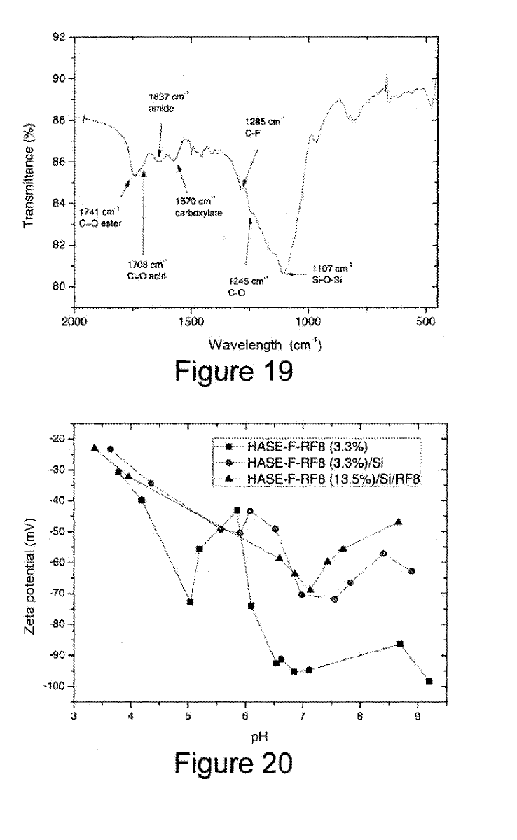

FIG. 19 represents the FT-IR (Fourier-transform infrared spectroscopy) spectrum of HASE-F-RF8/Si/RF8 in neutral medium;

FIG. 20 represents the aggregation of the silica nanoparticles characterized by the zeta potential, according to pH for HASE-F-FR8 (3.3%) (squares), HASE-F-RF8/Si (3.3%) (circles) and HASE-F-RF8 (13.5%)/Si/RF8 (triangles);

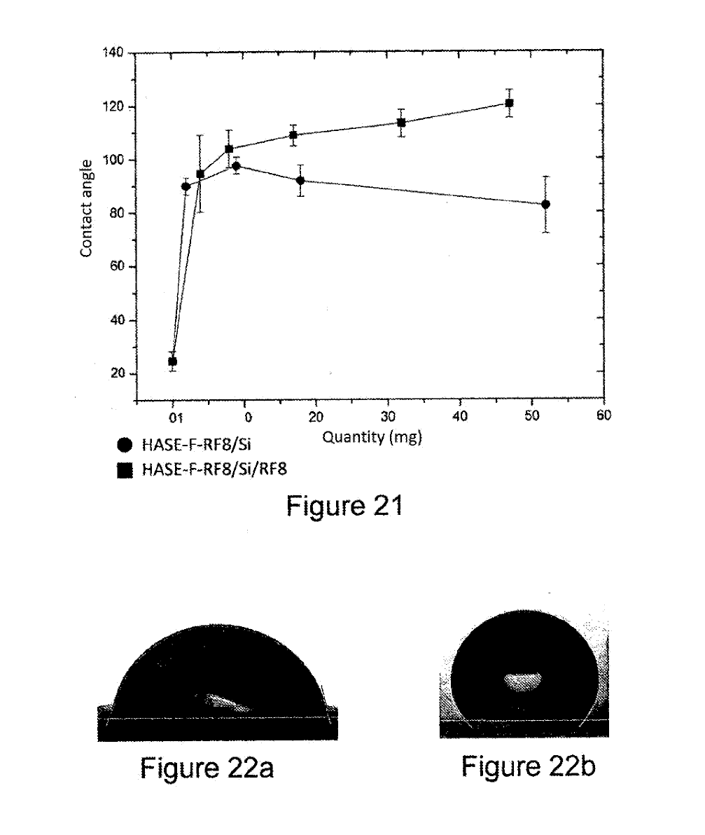

FIG. 21 is a graph illustrating the wettability with olive oil of HASE-F-RF8/Si (circles) and HASE-F-RF8/Si/RF8 (squares) according to the quantity (mg) smeared on a glass plate;

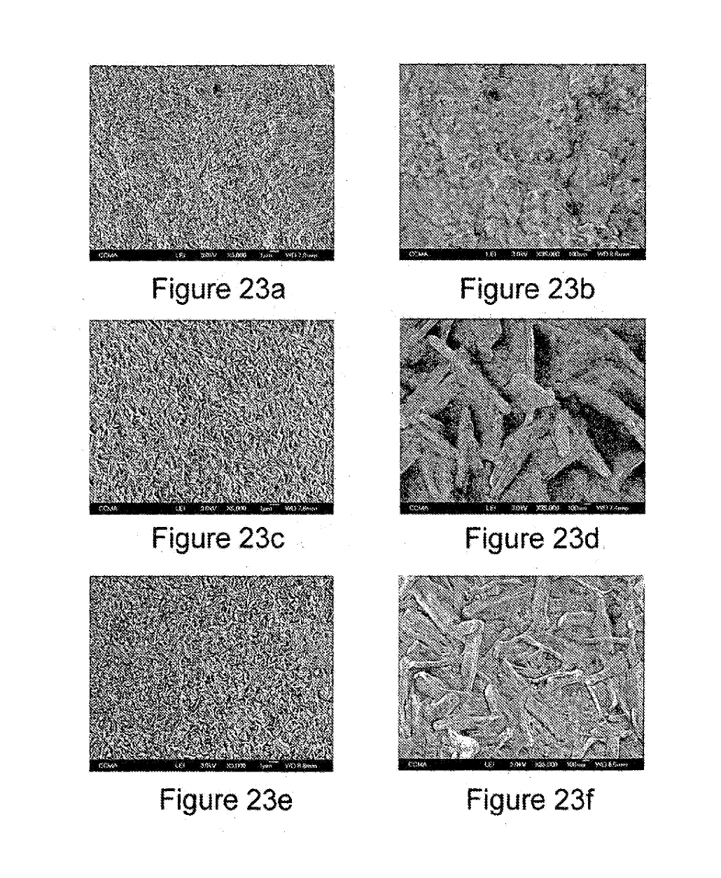

FIGS. 22a and 22b represent images of the contact angle of the compounds HASE-F-RF8/Si (FIG. 22a) and HASE-F-RF8/Si/RF8 (FIG. 22b) using the smearing method with, respectively, 52 mg and 47 mg on glass plates;

FIGS. 23a to 23f represent SEM (Scanning Electron Microscopy) images of HASE-F-RF8/Si using smearing (FIGS. 23a and 23b, with 52 mg deposited) and HASE-F-RF8/Si/RF8 using smearing (FIGS. 23c and 23d with 47 mg deposited) and spraying (FIGS. 23e and 23f with 15 mg deposited);

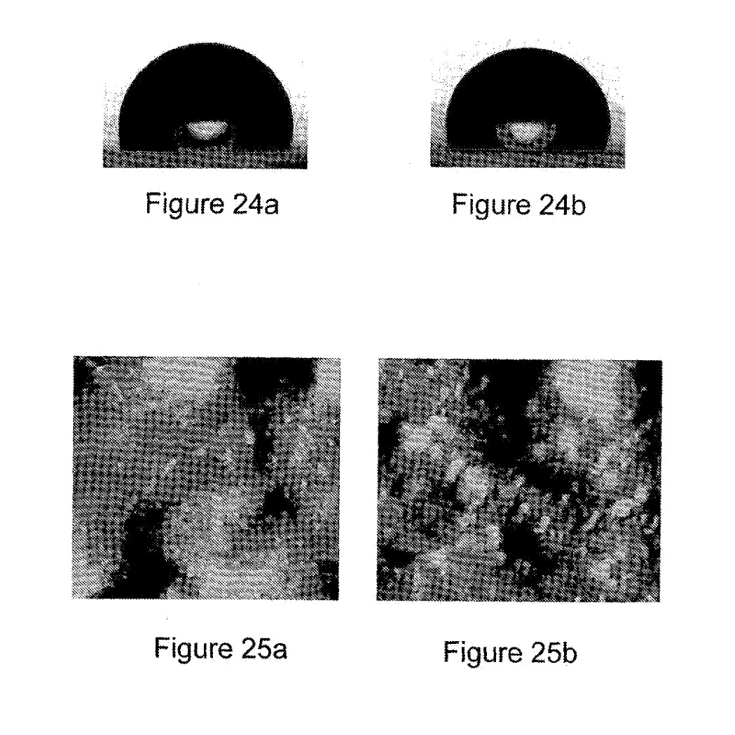

FIGS. 24a and 24b represent images of the contact angle for HASE-F-RF8/Si/RF8 using the smearing (FIG. 24a) and spraying (FIG. 24b) methods with 15 mg deposited on a glass plate;

FIGS. 25a and 25b illustrate the dispersion of HASE-F-RF8/Si/RF8 using the smearing (FIG. 25a) and spraying (FIG. 25b) methods;

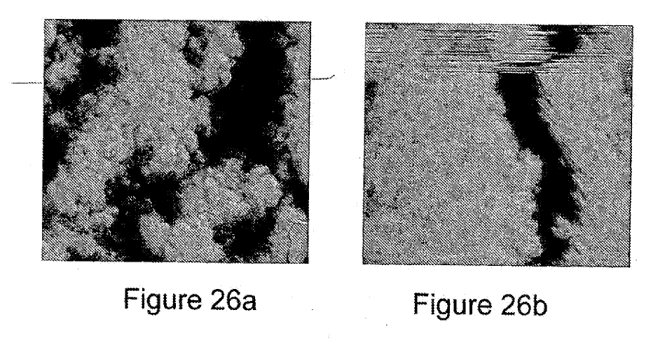

FIGS. 26a and 26b represent AFM (Atomic Force Microscopy) images of smear depositions of the compounds HASE-F-RF8/Si/RF4 for 49 mg (FIG. 26a) and HASE-F-RF8/Si/RF6 for 60 mg (FIG. 26b).

The compound according to the invention is a compound formed by amine functionalized micro- or nanoparticles, covalently associated with rheology-modifying polymers, characterized in that:
the functionalized micro- or nanoparticles are amine functionalized micro- or nanoparticles of aluminum oxide (Al₂O₃), copper oxide (CuO), iron oxide (Fe₃O₄ or γ-Fe₂O₃), magnesium oxide (MgO), silica (SiO₂), titanium dioxide (TiO₂) or zinc oxide (ZnO), having a nominal diameter between 1 and 1500 nm;
the rheology-modifying or -adapting polymers are chosen from non-associative polymers or associative polymers.

According to the invention, a nanoparticle is defined as being a nano-object wherein the three dimensions are on a nanometric scale, i.e. a particle wherein the nominal diameter is less than 100 nm.

Preferably, the nanoparticle according to the invention has a diameter between 1 and 50 nm. More preferably, the nominal diameter is between 5 nm and 25 nm.

A microparticle according to the invention is a micro-object wherein the three dimensions are on a micrometric scale, i.e. a particle wherein the nominal diameter is between 100 nm and 100,000 nm. Preferably, the microparticle according to the invention has a nominal diameter between 100 nm and 5000 nm. More preferably, the nominal diameter of the microparticle is between 100 nm and 1500 nm.

The micro- or nanoparticles may be produced using various methods, notably by vapor phase, liquid phase, solid medium, composite medium chemical synthesis or by means of physicochemical methods such as evaporation/condensation.

According to one preferred embodiment of the invention, the micro- or nanoparticles of aluminum oxide (Al₂O₂), magnesium oxide (MgO), silica (SiO₂), titanium dioxide (TiO₂), zinc oxide (ZnO) or copper oxide (CuO) may be synthesized in a composite medium using the sol-gel method. The principle of the sol-gel method is based on the use of a series of hydrolysis-condensation reactions, at a moderate temperature close to ambient temperature, to prepare oxide lattices which may in turn be heat-treated. The soluble metal species may also contain organic constituents which may be adjusted according to the applications. The first step in sol-gel synthesis is hydroxylation of the metal alkoxy, which occurs during the hydrolysis of the alkoxy group:

Step 1: Hydrolysis:

M′-OR′+H₂O→M′-OH+R′OH

where M′ represents Aluminum, Copper, Magnesium, Silicon or Titanium; and R′ represents an alkyl organic group comprising 1 to 5 carbon atoms, preferentially 2 to 3 carbon atoms.

The hydroxyl reagent groups are then generated. The solution obtained is referred to as a sol. They are then modified with polycondensation reactions via two competitive mechanisms: the formation of an oxygen bridge (oxolation) and a hydroxo bridge (olation). This corresponds to the formation of the mineral macromolecular lattice with elimination of water or alcohol:

Step 2: Condensation:

Oxolation: M′-OH+R′OH→M′-O-M′+R′OH

where M′ and R′ are as defined above, i.e. M′ represents Aluminum, Copper, Magnesium, Silicon or Titanium; and R′ represents an alkyl organic group comprising 1 to 5 carbon atoms, preferentially 2 to 3 carbon atoms.

Olation: M′-OH+HO-M′→M′(OH)₂M′

where M′ represents Aluminum, Copper, Magnesium, Silicon or Titanium.

The gel corresponds to the formation of a three-dimensional lattice of Van der Waals' bonds.

The micro- or nanoparticles suitable for use according to the invention preferentially have a mean nominal diameter between 1 and 1500 nm.

Advantageously, the micro- or nanoparticles of aluminum oxide (Al₂O₃) have a mean nominal diameter between 5 and 400 nm. Preferably, the mean nominal diameter thereof is between 10 and 200 nm. More preferably, the mean nominal diameter thereof is 13, 20 or 50 nm.

Advantageously, the micro- or nanoparticles of copper oxide (CuO) have a mean nominal diameter between and 100 nm. Preferably, the mean nominal diameter thereof is between 30 and 80 nm. More preferably, the mean nominal diameter thereof is between 40 and 70 nm.

Advantageously, the micro- or nanoparticles of iron oxide (Fe₃O₄ or γ-Fe₂O₃) have a mean nominal diameter between 1 and 50 nm. Preferably, the mean nominal diameter thereof is between 5 and 30 nm. More preferably, the mean nominal diameter thereof is between 10 and 30 nm.

Advantageously, the micro- or nanoparticles of magnesium oxide (MgO) have a mean nominal diameter between 20 and 100 nm. Preferably, the mean nominal diameter thereof is between 40 and 70 nm. More preferably, the mean nominal diameter thereof is between 50 and 60 nm.

Advantageously, the micro- or nanoparticles of silica (SiO₂) have a mean nominal diameter between 5 and 500 nm. Preferably, the mean nominal diameter thereof is between 10 and 450 nm. More preferably, the micro- or nanoparticles of silica have a mean nominal diameter of 14, 22, 292 or 448 nm.

Advantageously, the micro- or nanoparticles of titanium oxide (TiO₂) have a mean nominal diameter between 1 and 300 nm. Preferably, the mean nominal diameter thereof is between 5 and 150 nm. More preferably, the mean nominal diameter thereof is between 10 and 25 nm.

Advantageously, the micro- or nanoparticles of zinc oxide (ZnO) have a mean nominal diameter between 5 and 500 nm. Preferably, the mean nominal diameter thereof is between 20 and 200 nm. More preferably, the mean nominal diameter thereof is between 50 and 60 nm. According to the invention, the micro- or nanoparticles are functionalized.

The micro- or nanoparticles may be functionalized for example by primary or secondary amine functions, epoxy functions, alcohol functions and thiol functions.

Preferably, the micro- or nanoparticles of aluminum oxide (Al₂O₃), copper oxide (CuO), iron oxide (Fe₃O₄ or γ-Fe₂O₃), magnesium oxide (MgO), silica (SiO₂), titanium dioxide (TiO₂) or zinc oxide (ZnO) are amine functionalized.

Preferably, the micro- or nanoparticles are amine functionalized micro- or nanoparticles of inorganic or metal oxide notably aluminum oxide (Al₂O₂), copper oxide (CuO), iron oxide (Fe₃O₄ or γ-Fe₂O₃), magnesium oxide (MgO), silica (SiO₂), titanium dioxide (TiO₂) or zinc oxide (ZnO).

The general diagram of the amine functionalization of the micro- or nanoparticles is illustrated in FIGS. 2b and 3.

The details of a synthesis process of amine functionalized 292 nm and 448 nm silica microparticles are described in example 1. Example 2 describes a synthesis process of amine functionalized 14 nm silica nanoparticles. Example 3 describes the synthesis of amine functionalized titanium dioxide micro- or nanoparticles.

Preferably, the amine function content of the amine functionalized micro- or nanoparticles is between 0.1 and 10 meq/g of micro- or nanoparticles.

The number of NH2 functions obtained is in meq/g of micro- or nanoparticles and is calculated according to the following equation:

$$Number of {NH}_{2} functions ( \frac{meq}{g} ) = \frac{\% N mass ( determined by elemental analysis )}{Molar mass of nitrogen} \times 100$$

Preferably, the amine function content of the amine functionalized micro- or nanoparticles is between 0.5 and 5 meq/g of micro- or nanoparticles. More preferably, the amine function content of the amine functionalized micro- or nanoparticles is approximately 1 or 2.5 meq/g of micro- or nanoparticles.

As described above, the micro- or nanoparticles according to the invention are amine functionalized, in order to react on the acid functions of the polymers.

The amine functionalized micro- or nanoparticles according to the invention are preferentially amine functionalized micro- or nanoparticles of silica (SiO₂) or titanium dioxide (TiO₂).

The rheology-modifying or adapting polymers according to the invention are already used, alone, for the rheological properties thereof in fields such as cosmetics or paint.

They consist of hydrocarbon or fluorocarbon polymers synthesized for example by means of polymerization in emulsion.

They are then characterized by infrared (IR) using the KBr pellet method, by goniometry following dry deposition on a glass surface, but also in solution by means of nuclear magnetic resonance (NMR) and rheology. The polymers according to the invention also contribute to the stability of the formulations or protective topical agents.

The polymers suitable for use belong to two different classes: non-associative polymers and associative polymers.

Non-associative polymers or “Alkali-Swellable Emulsions” (ASE) suitable for use according to the invention are already widely used, alone, as thickeners in latex coatings, paints and adhesives.

They essentially consist of monomers of acrylic or methacrylic acid and C1-C4 alkyl acrylate, preferentially ethyl acrylate.

They are generally synthesized by polymerization in emulsion in acidic aqueous medium (pH less than 4) and are obtained in the form of colloidal suspension of polymers (or synthetic latexes). The acid functions of the copolymer are ionized in a basic medium, inducing the solubilization and swelling of the polymer (increase in hydrodynamic volume). The ethyl acrylate groups are relatively blocked to induce hydrophobic associations between polymer chains and increase the viscosity.

Furthermore, the ASE non-associative polymers may be optimized by cross-linking. The cross-linking phenomenon makes it possible to physically increase the density of the polymer lattice which reduces the possibility of molecular motion and thus increases viscosity.

The non-associative polymers according to the invention may comprise a hydrocarbon chain (ASE-H) and/or a fluorocarbon chain (ASE-F).

Preferably, the ASE-H non-associative polymers comply with the following general formula (I):

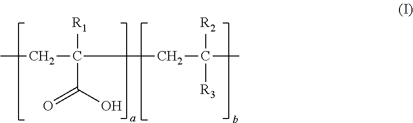

wherein:
R1 and R2 represent a hydrogen atom or a methyl group —CH₃;
R3 represents [Q]_{d1}-(CH₂)ₙ—H wherein n is between 1 and 30, d1 corresponds to 0 or 1, and Q corresponds to —C(O)—O or —C(O)—NH—;

or

R3 represents [Q]_{d2}-α, wherein:
d2 corresponds to 0 or 1;
Q corresponds to —C(O)—O or —C(O)—NH—; and
α corresponds to —C(CH₃)₃; —CH(CH₃)₂; —C(CH₃)₂—CH₂—C(CH₃)₃; —CN; —CH₂CH₂—N+(CH₃)₂(CH₂CO₂⁻); —CH₂CH₂—NH—C(CH₃)₃; —CH₂CH₂—N(CH₃)₂; pyrrolidinone; caprolactam;

and wherein the values a and b are integers, identical or different, greater than 1. Preferentially, a is between 1 and 10,000 and b is between 1 and 20,000.

Throughout the description, the polymers are compounds of different monomers or macromers, at given molar concentrations, varying according to the values of a, b and/or c. Obviously, the sequence of the various monomers or macromers in the polymers obtained is variable and is not fixed in formulas (I), (II), (III), (V), (VI), (VII) and (VIII).

More preferably, the hydrocarbon-chain ASE polymers (ASE-H) comprise acrylic and/or methacrylic acid and C1-C4 alkyl acrylate monomers.

Advantageously, the hydrocarbon-chain ASE polymers (ASE-H) comprise:
5 to 50% mol. acrylic and/or methacrylic acid;
50 to 95% mol. C1-C4 alkyl acrylates.

More preferentially, the ASE-H polymers comprise the following monomers:
10 to 20% mol. methacrylic acid (MA);
80 to 90% mol. ethyl acrylate (EA).

Hydrocarbon-chain ASE polymers (ASE-H) have particularly advantageous thickening properties when the methacrylic acid/ethyl acrylate ratio is between 0.1 and 0.5. The more preferable ratio is 0.21.

As mentioned above, the ASE non-associative polymers according to the invention may also comprise a fluorocarbon chain (ASE-F).

Preferably, the ASE-F non-associative polymers comply with the following general formula (II):

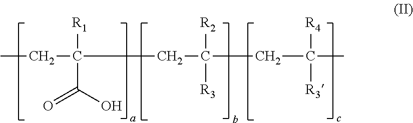

wherein:
R1, R2 and R4 represent a hydrogen atom or a methyl group —CH₃;
R3 represents [Q]_{d1}-(CH₂)ₙ—H wherein n is between 1 and 30, d1 corresponds to 0 or 1, and Q corresponds to —C(O)—O or —C(O)—NH—;

or

R3 represents [Q]_{d2}-α, wherein:
d2 corresponds to 0 or 1;
Q corresponds to —C(O)—O or —C(O)—NH—; and
α corresponds to —C(CH₃)₃; —CH(CH₃)₂; —C(CH₃)₂—CH₂—C(CH₃)₃; —CN; —CH₂CH₂—N+(CH₃)₂(CH₂CO₂⁻); —CH₂CH₂—NH—C(CH₃)₃; —CH₂CH₂—N(CH₃)₂; pyrrolidinone; caprolactam;
R3′ represents [Q]_{d1}-(CH₂)ₙ—(CX₂)ₚX wherein n is between 1 and 30, d1 corresponds to 0 or 1, Q corresponds to —C(O)—O or —C(O)—NH—, X is a fluorine atom F and p is between 1 and 12;

and wherein the values a and c are integers, identical or different, greater than 1 and b is greater than or equal to 0; preferentially, a is between 1 and 10,000, b is between 1 and 5,000 and c is between 1 and 8,000.

These ASE-F polymers preferentially consist of monomers of:
methacrylic acid (MA),
2,2,2-trifluoroethyl methacrylate (TEEM) or trifluoroethyl acrylate or 2-perfluorobutylethyl acrylate or 2-perfluorohexylethyl acrylate or 2-perfluorooctylethyl acrylate; and
C1-C4 alkyl acrylate, preferentially ethyl acrylate (EA).

Introducing a fluorocarbon chain makes it possible, for cosmetic or pharmaceutical use, to reduce surface uptake of toxins.

Preferably, the fluorocarbon-chain ASE polymers (ASE-F) consist of the following monomers:
20 to 75% mol. acrylic and/or methacrylic acid;
0 to 30% mol. C1-C4 alkyl acrylates; and
10 to 55% mol. 2,2,2-trifluoroethyl methacrylate (TFEM) or trifluoroethyl acrylate or 2-perfluorobutylethyl acrylate or 2-perfluorohexylethyl acrylate or 2-perfluorooctylethyl acrylate.

More preferably, the fluorocarbon-chain ASE polymers (ASE-F) comprise monomers of methacrylic acid (MA), ethyl acrylate (EA) and 2,2,2-trifluoroethyl methacrylate (TFEM).

Advantageously, the hydrocarbon-chain ASE polymers (ASE-F) comprise:
50 to 60% mol. methacrylic acid (MA);
5 to 15% mol. ethyl acrylate (EA); and 30 to 40% mol. 2,2,2-trifluoroethyl methacrylate (TFEM).

Even more preferentially, the fluorocarbon-chain ASE polymers (ASE-F) have particularly advantageous thickening properties when the ratio of the methacrylic acid (MA)/ethyl acetate (AE) ratio is between 7 and 0.1. A general formula of ASE-H and ASE-F polymers suitable for use according to the invention is illustrated in FIG. 4. A synthesis process of ASE-H and ASE-F polymers is detailed in example 4, and is summarized in FIG. 4, wherein the values a, b and c are integers, identical or different, greater than 1. Preferentially, a is between 1 and 10,000; b is between 1 and 5,000 and c is between 1 and 8,000.

The associative polymers suitable for use according to the invention consist of a hydrophilic macromolecular structure whereon hydrophobic groups are found. These hydrophobic groups are frequently short-chain (having between 1 and 6 carbon atoms) or long-chain (having more than 6 carbon atoms) alkyl links capable of forming aggregates, clusters, of the micellar type from a so-called critical aggregation concentration. These aggregates are referred to as hydrophobic junctions.

To date, three types of different associative polymers, suitable for use according to the invention, are marketed; they consist of:
hydrophobically modified ethylene-oxide urethane (HEUR);
cellulose derivatives; and hydrophobically modified alkali-swellable emulsions (HASE).

These three types of polymers are classified into two categories of associative polymers according to the molecular architecture thereof:

1. so-called telechelic polymers; or

2. comb type polymers.

The schematic representation of telechelic and comb type compounds is illustrated in FIG. 1. Telechelic polymers (HEUR) are linear chains of polymers containing chain-end hydrophobic groups. The comb type polymers (cellulose derivatives and HASE polymers) are polymers containing hydrophobic chains along the skeleton.

The HASE polymers are generally copolymers of methacrylic acid (MA), ethyl acrylate (EA) and a quantity of hydrophobic groups, which are macromonomers or macromers.

Despite the presence of hydrophobic groups, which are long hydrocarbon chains, HASE polymers are soluble in aqueous medium, rendering these compounds of particular interest.

As for ASE polymers, HASE polymers are generally prepared by means of polymerization in emulsion at a low pH, making it possible to obtain polymers having molar masses between 300,000 and 1,800,000 g/mol.

The rheological properties of HASE polymers have demonstrated that the viscosity was significantly pH-dependent. In the pH range between 2.4 and 4.5, the polymer skeleton bends to form a compact coil due to the poor quality of the solvent. The polymer solution is milky and consists of insoluble colloidal particles. At pH 6, the viscosity increases suddenly and then remains constant to pH 11, the carboxyl groups on the polymer skeleton are dissolved and the solution becomes transparent. The polymer chain then resembles a polyelectrolyte which induces the extension of the polymer skeleton due to mutual carboxylate repulsion and increased hydrodynamic volume. At the same time, a large number of inter- and intramolecular associations between the hydrophobic groups are formed, inducing the construction of a lattice in the aqueous medium.

At a basic pH, HASE polymers combine the properties of polyelectrolytes and the properties of non-charged associative polymers. Above pH 11, the viscosity decreases slowly due to the charge protection effect. Further factors may vary the dynamic nature of the polymer and the structure of the HASE polymers, such as notably the salt concentration in the medium. If this increases, the viscosity decreases significantly. The negative effect of adding salts may be compensated by adding a surfactant. The surfactant concentration may vary the viscosity of the medium. An increase in the concentration with a non-ionic surfactant increases the viscosity of the medium, whereas an anionic surfactant increases the viscosity up to a critical concentration where it drops.

The viscosity of the medium may also be reduced when the temperature increases.

The use of a strong base (NaOH) for neutralizing an HASE polymer induces the degradation thereof after a period of four weeks (pH=9.5). The use of a weak organic base (1-amino-1-methylpropanol) induces stability of the rheological properties of the solution for six weeks (pH=9.5).

Monovalent neutralizing agents may also be recommended. Indeed, in the case of the use of a di- or trivalent base molecule thus having the ability to neutralize more than one carboxylic acid function, a reduction in the ability of the polymer to unwind and extend completely may arise.

Finally, adding an organic solvent may reduce the viscosity of the medium by breaking the hydrophobic associations within the aqueous medium and solubilizing the hydrophobic groups.

Preferably, the polymers according to the invention are macromeric HASE polymers having a hydrocarbon chain (HASE-H-RH or HASE-F-RH) or fluorocarbon chain (HASE-F-RF).

The macromeric HASE associative polymers having a hydrocarbon chain (HASE-H-RH or HASE-F-RH) or a fluorocarbon chain (HASE-F-RF) according to the invention comply with the following general formula (III):

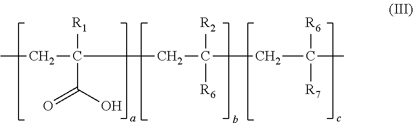

wherein:
R1, R2 and R6 represent a hydrogen atom or a methyl group;
R5 represents [Q]_{d1}-(CH₂)ₙ—(CX₂)ₚX wherein n is between 1 and 30, d1 corresponds to 0 or 1, Q corresponds to —C(O)—O or —C(O)—NH—; and:
if X is a hydrogen atom, then p is equal to 0;
if X is a fluorine atom, then p is between 1 and 12;

or

R5 represents [Q]_{d2}-α, wherein:
d2 corresponds to 0 or 1;
Q corresponds to —C(O)—O or —C(O)—NH—; and
α corresponds to —C(CH₃)₃; —CH(CH₃)₂; —C(CH₃)₂—CH₂—C(CH₂)₃; —CN; —CH₂CH₂—N+(CH₃)₂(CH₂CO₂⁻); —CH₂CH₂—NH—C(CH₃)₃; —CH₂CH₂—N(CH₃)₂; pyrrolidinone; caprolactam;
   R7 represents -[Q]_{d3}-(OCH₂CH₂)_{q}-[Q″]_{d4}-(CH₂)ₙ(CX₂)ₚX wherein Q′ corresponds to —CH₂, C(O), O—C(O) or —NH—C(O), n is between 1 and 30, q is between 1 and 150, d3 and d4 correspond to 0 and/or 1, Q″ corresponds to —O—C(O) or —NH—C(O); and:
if X is a hydrogen atom, then p is equal to 0;
if x is a fluorine atom, then p is between 1 and 12;

and wherein the values a and c are integers, identical or different, greater than or equal to 1 and b is greater than or equal to 0.

Preferentially, a is between 1 and 10,000; b is between 1 and 5,000.

Preferably, the hydrocarbon-chain HASE-H-RH polymers consist of monomers of methacrylic acid (MA), C1-C4 alkyl acrylates and a macromer which is an ester having the general formula (IV):

CH₂═CH(CH₃)—C(O)(OCH₂CH₂)_{q}OC(OC)(CH₂)ₙ—H (IV)
wherein q denotes a number between 5 and 10 and n is between 6 and 30 carbon atoms.

More preferably, the HASE-H-RH polymers comprise monomers of methacrylic acid (MA), ethyl acrylate (EA), and a macromer which an ester having a general formula (IV) as defined above, and thus comply with the following general formula (V):

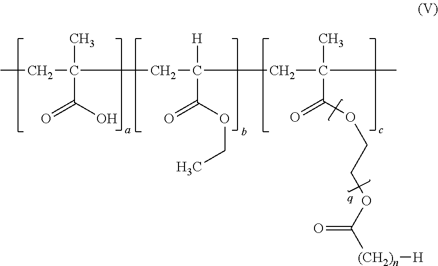

wherein:
q denotes a number between 5 and 10;
n is between 1 and 30;
a and c are integers, identical or different, greater than or equal to 1, and b is greater than or equal to 0; preferentially, a is between 1 and 10,000, b is between 1 and 10,000 and c is between 1 and 5,000.

More preferably, the HASE-H-RH polymers comply with general formula (V) and comprise:
5 to 85% mol. methacrylic acid (MA);
5 to 60% mol. ethyl acrylate (EA); and
1 to 90% mol. of a macromer which is an ester having general formula (IV) defined above.

The particularly preferred HASE-H-RH polymers comply with formula (V) above, and are such that:
q is equivalent to 7 and n is equal to 6 carbon atoms (hereinafter referred to as HASE-H-RH4 polymer);
q is equivalent to 7 and n is equal to 8 carbon atoms (hereinafter referred to as HASE-H-RH6 polymer);
q is equivalent to 9 and n is equal to 10 carbon atoms (hereinafter referred to as HASE-H-RH8 polymer).

Alternatively, according to the invention, the ethyl acrylate (EA) monomer of the HASE-H-RH polymer of formula (V) above may be replaced by a monomer of 2,2,2-trifluoroethyl methacrylate (TFEM) and thus corresponds to an HASE-F-RH polymer complying with the following general formula (VI);

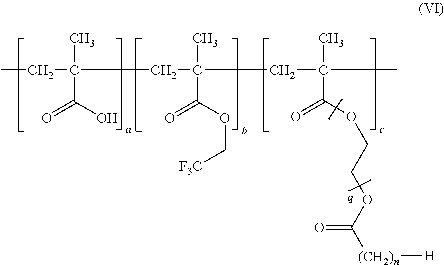

wherein:
q denotes a number between 5 and 10;
n is between 6 and 30 carbon atoms;
a and c are integers, identical or different, greater than or equal to 1, and b is greater than or equal to 0; preferentially, a is between 1 and 10,000, b is between 1 and 10,000 and c is between 1 and 5,000.

More preferably, the HASE-F-RH polymers comply with general formula (VI) above and comprise:
30 to 85% mol. methacrylic acid (MA);
0 to 50% mol. 2,2,2-trifluoroethyl methacrylate (TFEM); and
1 to 90% mol. of a macromer which is an ester having general formula (IV).

The particularly preferred HASE-F-RH polymers comply with formula (VI) above, and are such that:
q is equivalent to 7 and n is equal to 6 carbon atoms (hereinafter referred to as HASE-F-RH4 polymer);
q is equivalent to 7 and n is equal to 8 carbon atoms (hereinafter referred to as HASE-F-RH6 polymer);
q is equivalent to 9 and n is equal to 10 carbon atoms (hereinafter referred to as HASE-F-RH8 polymer).

Advantageously, the Applicant further succeeded in demonstrating that substituting hydrocarbon links with fluorocarbons on the macromer was possible in an HASE skeleton.

By modifying the skeleton thereof with fluorocarbon macromers in this way, the rheology-modifying polymer according to the invention can disperse the micro- or nanoparticles while providing the hydrophobicity and oleophobicity required for protection against chemical agents.

The structure of such fluorocarbon-chain HASE polymers (HASE-F), also suitable for use according to the invention, complies with the following general formula (VII):

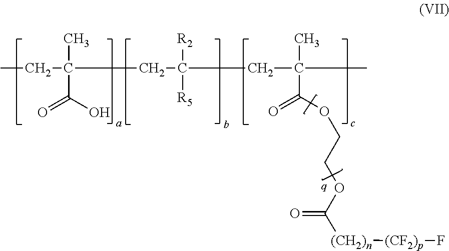

wherein:
R2 represents a hydrogen atom or a methyl group;
R5 represents [Q]_{d1}-(CH₂)ₙ—(CX₂)ₚX wherein n is between 1 and 30, d1 corresponds to 0 or 1, Q corresponds to —C(O)—O or —C(O)—NH—; and:
if X is a hydrogen atom, then p is equal to 0;
if X is a fluorine atom, then p is between 1 and 12;

or R5 represents [Q]_{d2}-α, wherein:
d2 corresponds to 0 or 1;
Q corresponds to —C(O)—O or —C(O)—NH—; and
α corresponds to —C(CH₃)₃; —CH(CH₃)₂; —C(CH₃)₂—CH₂—C(CH₃)₃; —CN; —CH₂CH₂—N+(CH₃)₂(CH₂CO₂⁻); —CH₂CH₂—NH—C(CH₃)₃; —CH₂CH₂—N(CH₃)₂; pyrrolidinone; caprolactam;
q denotes a number between 1 and 150;
n is an integer between 1 and 30;
p is an integer between 1 and 12;

and wherein the values a and c are integers, identical or different, greater than or equal to 1, and b is greater than or equal to 0; preferentially, a is between 1 and 10,000, b is between 1 and 10,000 and c is between 1 and 5,000.

More preferably, the HASE-F polymers comply with the following general formula (VIII):

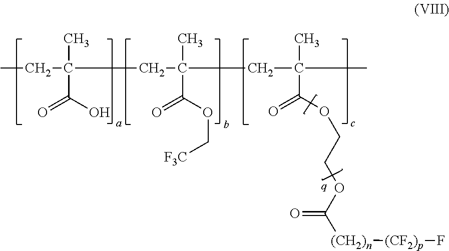

wherein:
q denotes a number between 5 and 10;
n is an integer between 1 and 30;
p is an integer between 1 and 12;
a and c are integers, identical or different, greater than or equal to 1, and b is greater than or equal to 0; preferentially, a is between 1 and 10,000, b is between 1 and 10,000 and c is between 1 and 5,000.

Even more preferentially, the HASE-F polymers comply with the general formula (VIII) above wherein:
q is equivalent to 5, 7 or 9;
n is equivalent to 2;
p is equivalent to 4, 6 or 8; and
a and c are integers, identical or different, greater than or equal to 1, and b is greater than or equal to 0; preferentially, a is between 1 and 10,000, b is between 1 and 10,000 and c is between 1 and 5,000. More preferably, the HASE-F polymers complying with general formula (VIII) comprise the following monomers:
5 to 85% mol. methacrylic acid (MA);
1 to 70% mol. 2,2,2-trifluoroethyl methacrylate (TFEM); and
1 to 50% mol. of a macromer which is an ester having general formula (IX):

CH₂═CH(CH₃)—C(O)(OCH₂CH₂)_{q}OC(O)(CH₂)₂—(CF₂)ₚF (IX)

wherein:
q is equivalent to 5, 7 or 9; and
p is equivalent to 4, 6 or 8.

The particularly preferred HASE-F polymers comply with formula (VIII) above, and are such that:
q is equivalent to 5 and p is equal to 4 carbon atoms (hereinafter referred to as HASE-F-RF4 polymer);
q is equivalent to 7 and p is equal to 6 carbon atoms (hereinafter referred to as HASE-F-RF6 polymer);
q is equivalent to 7 and p is equal to 6 carbon atoms (hereinafter referred to as HASE-F-RF8 polymer);

The HASE polymers described above may be synthesized using the same method as for ASE polymers. Details of a synthesis process of HASE-H and HASE-F polymers are given in example 5.

Advantageously, the rheology-modifying or adapting polymers suitable for use in the compounds according to the invention are chosen from:

i) the ASE-H non-associative polymers having the following general formula (I):

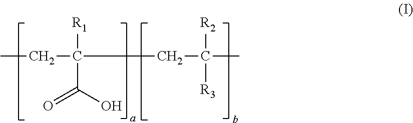

wherein:
R1 and R2 represent a hydrogen atom or a methyl group —CH₃;
R3 represents [Q]_{d1}-(CH₂)ₙ—H wherein n is between 1 and 30, d1 corresponds to 0 or 1, and Q corresponds to —C(O)—O or —C(O)—NH—;

or

R3 represents [Q]_{d2}-α, wherein:
d2 corresponds to 0 or 1;
Q corresponds to —C(O)—O or —C(O)—NH—; and
α corresponds to —C(CH₃)₃; —CH(CH₃)₂; —C(CH₃)₂—CH₂—C(CH₃)₃; —CN; —CH₂CH₂—N+(CH₃)₂(CH₂CO₂⁻); —CH₂CH₂—NH—C(CH₃)₃; —CH₂CH₂—N(CH₃)₂; pyrrolidinone; caprolactam;

and wherein the values a and b are integers, identical or different, greater than 1;

or

ii) the ASE-F non-associative polymers having the following general formula (II):

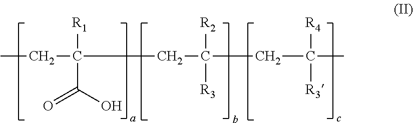

wherein:
R1, R2 and R4 represent a hydrogen atom or a methyl group —CH₃;
R3 represents [Q]_{d1}-(CH₂)ₙ—H wherein n is between 1 and 30, d1 corresponds to 0 or 1, and Q corresponds to —C(O)—O or —C(O)—NH—;

or

R3 represents [Q]_{d2}-α, wherein:
d2 corresponds to 0 or 1;
Q corresponds to —C(O)—O or —C(O)—NH—; and
α corresponds to —C(CH₃)₃; —CH(CH₃)₂; —C(CH₃)₂—CH₂—C(CH₃)₃; —CN; —CH₂CH₂—N+(CH₃)₂(CH₂CO₂⁻); —CH₂CH₂—NH—C(CH₃)₃; —CH₂CH₂—N(CH₃)₂; pyrrolidinone; caprolactam;
R3′ represents [Q]_{d1}-(CH₂)ₙ—(CX₂)ₚX wherein n is between 1 and 30, d1 corresponds to 0 or 1, Q corresponds to —C(O)—O or —C(O)—NH—, X is a fluorine atom F and p is between 1 and 12;

and wherein the values a and c are integers, identical or different, greater than 1 and b is greater than or equal to 0;

or

iii) the macromeric HASE associative polymers having a hydrocarbon chain (HASE-H-RH or HASE-F-RH) or a fluorocarbon chain (HASE-F-RF) complying with the following general formula (III):

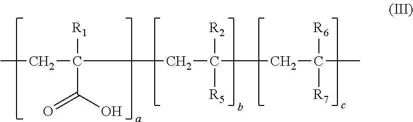

wherein:
R1, R2 and R6 represent a hydrogen atom or a methyl group;
R5 represents [Q]_{d1}-(CH₂)ₙ—(CX₂)ₚX wherein n is between 1 and 30, d1 corresponds to 0 or 1, Q corresponds to —C(O)—O or —C(O)—NH—; and:
if X is a hydrogen atom, then p is equal to 0;
if X is a fluorine atom, then p is between 1 and 12;

or

R5 represents [Q]_{d2}-α, wherein:
d2 corresponds to 0 or 1;
Q corresponds to —C(O)—O or —C(O)—NH—; and
α corresponds to —C(CH₃)₃; —CH(CH₃)₂; —C(CH₃)₂—CH₂—C(CH₃)₃; —CN; —CH₂CH₂—N+(CH₃)₂(CH₂CO₂⁻); —CH₂CH₂—NH—C(CH₃)₃; —CH₂CH₂—N(CH₃)₂; pyrrolidinone; caprolactam;
R7 represents [Q′]_{d3}-(OCH₂CH₂) [Q′″]_{d4}-(CH₂)ₙ(CX₂)ₚX wherein Q′ corresponds to —CH₂, C(O), O—C(O) or —NH—C(O), n is between 1 and 30, q is between 1 and 150, d3 and d4 correspond to 0 and/or 1, Q″ corresponds to —O—C(O) or —NH—C(O); and:
if X is a hydrogen atom, then p is equal to 0;
if X is a fluorine atom, then p is between 1 and 12;

and wherein the values a and c are integers, identical or different, greater than or equal to 1 and b is greater than or equal to 0.

Particularly advantageously, the rheology-modifying or adapting polymers are chosen from:
ASE-H polymers comprising the following monomers:
10 to 20% mol. methacrylic acid (MA);
80 to 90% mol. ethyl acrylate (EA);

or
hydrocarbon-chain ASE polymers (ASE-F) comprising the following monomers:
50 to 60% mol. methacrylic acid (MA);
5 to 15% mol. ethyl acrylate (EA); and
30 to 40% mol. 2,2,2-trifluoroethyl methacrylate (TFEM);
HASE-H-RH polymers complying with general formula (V) and comprising:
5 to 85% mol. methacrylic acid (MA);
5 to 60% mol. ethyl acrylate (EA); and
1 to 90% mol. of a macromer which is an ester having general formula (IV) defined above;
HASE-F-RH polymers complying with general formula (VI) and comprising:
30 to 85% mol. methacrylic acid (MA);
0 to 50% mol. 2,2,2-trifluoroethyl methacrylate (TFEM); and
1 to 90% mol. of a macromer which is an ester having general formula (IV).

Even more advantageously, the rheology-modifying or adapting polymers are chosen from the following polymers:
ASE-H;
ASE-F;
HASE-H-RH4;
HASE-H-RH6;
HASE-H-RH8;
HASE-F-RH4;
HASE-F-RH6;
HASE-F-RH8;
HASE-F-RF4;
HASE-F-RF6;
HASE-F-RF8.

Moreover, the Applicant succeeded in demonstrating, as described in example 6 that increasing the molar ratio of hydrocarbon or fluorine macromers in the HASE polymers makes it possible to increase the viscosity of the polymers.

In addition, preferably, the molar percentage of macromers in the HASE polymers is between 1 and 85% mol. More preferentially, the molar percentage is between 3 and 50% mol. of macromers.

Even more preferentially, the molar percentage is 13.5% mol. of macromers.

Characterizing the set of polymers in terms of rheology and goniometry made it possible to demonstrate that HASE-F-RF8 polymers containing 3.3%; 13.5% and 45.9% mol. of macromers were particularly preferred notably for the oleophobicity thereof and the viscosity of the solutions thereof.

Particularly advantageously, the polymer according to the invention is HASE-F-RF8 polymer, preferentially containing 13.5% mol. macromer.

The compound according to the invention is formed by covalently associating one or a plurality of amine functionalized micro- or nanoparticles as described above, with one or a plurality of rheology-modifying polymers as described above.

Furthermore, the compounds according to the invention further having free amine functions situated on the micro- or nanoparticles, it is possible to graft further molecules thereon. By way of non-limiting example of an additional molecule suitable for grafting, mention may be made of 4,4,5,5,6,6,7,7,7-nonafluoroheptanoic acid (RF4), 4,4,5,5,6,6,7,7,8,8,9,9,9-tridecafluorononanoic acid (RF6) and 4,4,5,5,6,6,7,7,8,8,9,9,10,10,11,11,11-heptadecafluorodecanoic acid (RF8).

These molecules are preferentially grafted, covalently, on the HASE-F-RF8/Si compound according to the invention.

In order to obtain an optimal protective effect against toxic agents, it is preferable to obtain a homogeneous dispersion of the micro- or nanoparticles within the polymeric matrix, by removing any aggregation.

Furthermore, the micro- or nanoparticles being in powder form, they may induce inflammation of the lungs by fixing thereon (by inhalation) or passage into the blood (by skin penetration). Also, in order to avoid this type of toxicity and in order to control dispersion, the Applicant covalently grafted the micro- or nanoparticles onto the polymers. This grafting may be carried out using the so-called “grafting to” method.

Covalent association of micro- or nanoparticles with polymers may be carried out by reacting amine functionalized micro- or nanoparticles on polymers having carboxylic acid functions (amidation reaction) in aqueous phase. Grafting of micro- or nanoparticles onto the polymers may be performed via esterification or via amidation.

Preferably, the grafting is performed by amidation which is a reaction wherein the yield is greater than esterification due to the greater nucleophilicity of nitrogen compared to alcohol.

Examples of grafting amine functionalized micro- or nanoparticles with associative or non-associative polymers are detailed in example 7.

As described above, before the grafting step, the micro- or nanoparticles of aluminum oxide (Al₂O₃), copper oxide (CuO), iron oxide (Fe₃O₄ or γ-Fe₂O₃), magnesium oxide (MgO), silica (SiO₂), titanium dioxide (TiO₂) or zinc oxide (ZnO) were synthesized and amine functionalized to react on the acid functions of the rheology-modifying or adapting polymers.

The covalent bond offers numerous advantages. It will make it possible firstly to prevent the penetration of micro- or nanoparticles via the respiratory tract and also through the skin into the human or animal body, and, secondly, control the dispersion of the micro- or nanoparticles within the matrix wherein the micro- or nanoparticles are bound. To enable same, the polymer, or polymeric matrix, contains compounds wherein the functions are suitable for reacting with the micro- or nanoparticles but which also be used in a topical agent.

According to the invention, the micro- or nanoparticles are bound covalently to a rheology-modifying polymer containing fluorinated monomers for an increased film-forming property, on one hand, and an increase in hydrophobicity and oleophobicity, on the other, in order to “repel” toxins. The aim is also that of providing an optimum film-forming property for surface protection by means of the various polymers, and a variable degree of interaction between the micro- or nanoparticles.

The micro- or nanoparticles, according to the nature thereof, will enable adsorption (for example for silica nanoparticles) or destruction (for example for titanium dioxide nanoparticles) by photodegradation of the toxins coming into contact with the film prior to the penetration thereof into the skin or into the substrate.

Advantageously, according to the invention, nanoparticles are preferred to microparticles. Indeed, the choice of nanoparticles is guided by the very large specific surface area thereof in relation to microparticles so as to increase the adsorption efficacy thereof. These nano-objects are, furthermore, transparent enabling the protection to remain invisible. This micro- or nanoparticulate array is suitable for dispersion in a basic aqueous medium due to the presence of carboxylic acid in the copolymer and can thus be readily integrated into a topical agent.

The Applicant also studied varying the number of equivalents of aluminum oxide (Al₂O₂), copper oxide (CuO), iron oxide (Fe₃O₄ or γ-Fe₂O₃), magnesium oxide (MgO), silica (SiO₂), titanium dioxide (TiO₂) or zinc oxide (ZnO) in the compounds according to the invention, i.e. the number of amine function equivalents borne by the amine functionalized micro- or nanoparticles of aluminum oxide (Al₂O₃), copper oxide (CuO), iron oxide (Fe₃O₄ or γ-Fe₂O₃), magnesium oxide (MgO), silica (SiO₂), titanium dioxide (TiO₂) or zinc oxide (ZnO) relative to the number of acid function equivalents borne by the polymer.

The Applicant thus succeeded in demonstrating, as described in example 13, that a number of equivalents, for example of silica, less than or equal to 1 is suitable for obtaining compounds having enhanced properties, for example in respect of dispersion and oleophobicity.

The ratio of polymer/micro- or nanoparticles was calculated using the number of acid function equivalents borne by the polymer according to the number of amine function equivalents borne by the micro- or nanoparticles (for 1 acid function equivalent contained in the polymer, 1 amine function equivalent was introduced), (where for 1 eq of acid functions, 0.3 eq of amine functions introduced).

Preferably, the number of equivalents of aluminum oxide (Al₂O₃), copper oxide (CuO), iron oxide (Fe₃O₄ or γ-Fe₂O₃), magnesium oxide (MgO), silica (SiO₂), titanium dioxide (TiO₂) or zinc oxide (ZnO) in the compounds according to the invention is between 0.05 and 1. More preferentially, it is between 0.3 and 0.8. Even more preferentially, the number of equivalents is 0.3.

The invention secondly relates to a protective topical agent comprising a compound according to the invention, in a pharmaceutically and/or cosmetically acceptable medium.

According to one particular embodiment of the invention, the protective topical agent further comprises one or a plurality of detoxifying agents and/or one or a plurality of additional polymers.

The detoxifying agents are non-toxic and dermatologically acceptable.

By way of limiting examples of detoxifying agents, mention may be made of benzoyl peroxide, zinc peroxide, magnesium monoperoxyphthalate, sodium perborate, sodium percarbonate, potassium permanganate, carbamide peroxide (urea peroxide), calcium peroxide, titanium dioxide, and sulfur-containing agents such as N-acetyl cysteine, perpropionic acid, magnesium peroxide or neutralizing agents such as zinc oxide, complexing agents such as etidronic acid and the tetrasodium salt thereof, 1-hydroxyethylenediamine(1,1-diphosponic) acid, sodium propionate, magnesium hydroxycarbonate, potassium nitrate or thioglycolic acid.

The percentage by mass of these detoxifying agents is preferentially between 0.001 and 60% of the total weight of the composition.

Furthermore, the protective topical agent according to the invention may also further comprise one or a plurality of additional polymers chosen from polyperfluoromethyl-isopropyl ether, dimethicone and vinyldimethicone copolymer, diethyleneglycol, adipic acid and glycerin copolymer, Polysilicone-8 and oleic/linoleic/linolenic acid polyglycerides, the role whereof consists of rendering the compositions more fluid or more pleasant to apply. Polyperfluoromethyl-isopropyl ether is notably marketed under the trade name Fomblin™ HC.

Dimethicone and vinyldimethicone polymer is notably marketed as Silicone Elastomer Blend DC9041™ Diethyleneglycol, adipic acid and glycerin copolymer is notably marketed as Lexorez 100™. Polysilicone-8 is notably marketed as Silicones Plus Polymer VS80Dry™. These additional polymers are introduced as a percentage by mass varying for example from 0.005% to 10% of the total weight of the composition. The dermatological and/or cosmetic composition according to the invention may further contain emollient agents, softening agents, preservatives or fragrances.

The protective topical agents may be presented in the form of gel, lotion, oil-in-water or water-in-oil emulsion, dispersion, milk, cream, ointment, foam, stick, spray, aerosols or any other form suitable for topical application.

The protective topical agents according to the invention are intended to be applied onto the skin, in the form of prevention and in expectation of potential contact with toxic chemical agents. They are applied in a sufficient layer onto the face and onto any parts of the body liable to be exposed to toxic chemical agents. The protective topical agents thus preferentially also contain a protective barrier base and one or a plurality of detoxifying agents, in order, on one hand, to delay the skin penetration of toxic chemical agents and/or, on the other, to neutralize same before they can reach the sites of action in a living body.

According to one particular embodiment, the compounds according to the invention are introduced into BariedermTech™ cream marketed by Uriage™ containing notably water, Poly-2p® complex consisting of pyrrolidone polymer and biomimetic phosphorylcholine polymer (Poly-2P™), Glycerin and alcohol.

The invention thirdly relates to a compound or a protective topical agent according to the invention used as a medicinal product.

Indeed, by acting on the skin barrier, the compound or the protective topical agent according to the invention has preventive properties in respect of human or animal diseases. As such, the compound or the protective topical agent according to the invention may also be used as a substance or composition suitable for use in humans or animals or suitable for being administered thereto, with a view to correcting or modifying the physiological functions thereof by applying a pharmacological, immunological or metabolic action. The compound or the protective topical agent according to the invention finds applications in human medicine, notably in dermatology, for preventing skin irritations or allergies.

The invention thus fourthly relates to a compound or a protective topical agent according to the invention, for use in the prevention of skin irritations or allergies.

The irritations may for example be skin irritations or allergies associated with at-risk occupational practices or home improvement activities.

The at-risk occupational practices include the use of chemical or biological hazard agents, for example in the hospital or military sector.

Home improvement activities include chemical for example painting, mechanics, gardening or furniture restoration activities.

The invention fifthly relates to the use of a compound or a protective topical agent according to the invention, for protection or decontamination of the skin, notably due to biological and/or chemical hazard agents.

Indeed, the compound or the protective topical agent may also be used for non-therapeutic applications, for example cosmetics, i.e. it finds an application as a protective epidermal barrier against external attacks.

As such, the invention also relates to the cosmetic use of the compound or the protective topical agent according to the invention, for skin protection against toxins of the organophosphate compound (OPC) family including pesticides (OPPs) and organophosphate neurotoxins (OPNs), or against vesicant agents such as sulfur or nitrogen yperites, or lewisite and phosphate oxime.

The invention also relates to the cosmetic use of the compound or the protective topical agent according to the invention, for protection against UVA and/or UVB ultraviolet radiation from a natural or artificial source.

For this use against UVA and/or UVB ultraviolet radiation, preferentially, the compound associates amine functionalized micro- or nanoparticles of titanium dioxide (TiO₂), in anatase form, with rheology-modifying or adapting polymers.

Finally, the invention further relates to methods for synthesizing the compounds according to the invention. Such synthesis methods are described in the examples hereinafter.

The preferred method for preparing a compound according to the invention comprises steps for:
mixing a coupling agent with a catalyst;
adding the mixture obtained to a solution of rheology-modifying or adapting polymers chosen from non-associative or associative polymers, in water;
stirring the reaction mixture;
adding functionalized micro- or nanoparticles of aluminum oxide (Al₂O₂), magnesium oxide (MgO), silica (SiO₂), titanium dioxide (TiO₂), zinc oxide (ZnO) or copper oxide (CuO) having a nominal diameter between 5 and 1500 nm, previously dispersed in aqueous phase in the reaction mixture;
purifying the reaction mixture by dialysis; and
retrieving the compound formed by one or a plurality of amine functionalized micro- or nanoparticles covalently associated with one or a plurality of rheology-modifying polymers.

Preferably, the coupling agent is N-(3-dimethylaminopropyl)-N′-ethylcarbodiimide hydrochloride (EDC) and the catalyst is N-hydroxysuccinimide (NHS).

Finally, the invention lastly relates to an associative or non-associative polymer as described above, as a rheology-modifying polymer.

The polymer according to the invention is a non-associative polymer or an associative polymer complying with the general formulas (I), (II) or (III) as detailed above.

As such, according to the invention, the polymer complies with the following general formula (I):

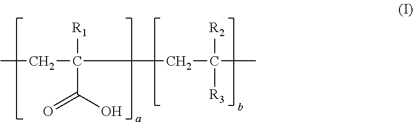

wherein:
R1 and R2 represent a hydrogen atom or a methyl group —CH₃;
R3 represents [Q]_{d1}-(CH₂)ₙ—H wherein n is between 1 and 30, d1 corresponds to 0 or 1, and Q corresponds to —C(O)—O or —C(O)—NH—;

or

R3 represents [Q]_{d2}-α, wherein:
d2 corresponds to 0 or 1;
Q corresponds to —C(O)—O or —C(O)—NH—; and
α corresponds to —C(CH₃)₃; —CH(CH₃)₂; —C(CH₃)₂—CH₂—C(CH₃)₃; —CN; —CH₂CH₂—N+(CH₃)₂(CH₂CO₂⁻); —CH₂CH₂—NH—C(CH₃)₃; —CH₂CH₂—N(CH₃)₂; pyrrolidinone; caprolactam;

and wherein the values a and b are integers, identical or different, greater than 1. Preferentially, a is between 1 and 10,000 and b is between 1 and 20,000;

or

complies with the following formula (II):

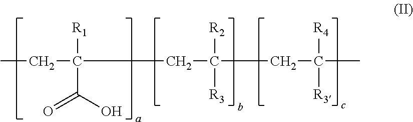

wherein:
R1, R2 and R4 represent a hydrogen atom or a methyl group —CH₃;
R3 represents [Q]_{d1}-(CH₂)ₙ—H wherein n is between 1 and 30, d1 corresponds to 0 or 1, and Q corresponds to —C(O)—O or —C(O)—NH—;

or

R3 represents [Q]_{d2}-α, wherein:
d2 corresponds to 0 or 1;
Q corresponds to —C(O)—O or —C(O)—NH—; and
α corresponds to —C(CH₃)₃; —CH(CH₃)₂; —C(CH₃)₂—CH₂—C(CH₃)₃; —CN; —CH₂CH₂—N+(CH₃)₂(CH₂CO₂⁻); —CH₂CH₂—NH—C(CH₃)₃; —CH₂CH₂—N(CH₃)₂; pyrrolidinone; caprolactam;
R3′ represents [Q]_{d1}-(CH₂)ₙ—(CX₂)ₚX wherein n is between 1 and 30, d1 corresponds to 0 or 1, Q corresponds to —C(O)—O or —C(O)—NH—, X is a fluorine atom F and p is between 1 and 12;

and wherein the values a and c are integers, identical or different, greater than 1 and b is greater than or equal to 0; preferentially, a is between 1 and 10,000, b is between 1 and 5,000 and c is between 1 and 8,000;

or complies with the following formula (III):

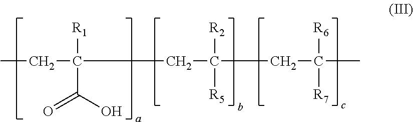

wherein:
R1, R2 and R6 represent a hydrogen atom or a methyl group;
R5 represents [Q]_{d1}-(CH₂)ₙ—(CX₂)ₚX wherein n is between 1 and 30, d1 corresponds to 0 or 1, Q corresponds to —C(O)—O or —C(O)—NH—; and:
if X is a hydrogen atom, then p is equal to 0;
if X is a fluorine atom, then p is between 1 and 12;

or

R5 represents [Q]_{d2}-α, wherein:
d2 corresponds to 0 or 1;
Q corresponds to —C(O)—O or —C(O)—NH—; and
α corresponds to —C(CH₃)₃; —CH(CH₃)₂; —C(CH₃)₂—CH₂—C(CH₃)₃; —CN; —CH₂CH₂—N+(CH₃)₂(CH₂CO₂⁻); —CH₂CH₂—NH—C(CH₃)₃; —CH₂CH₂—N(CH₃)₂; pyrrolidinone; caprolactam;
R7 represents -[Q′]_{d3}-(OCH₂CH₂)_{q}-[Q″]_{d4}-(CH₂)ₙ(CX₂)ₚX wherein Q′ corresponds to —CH₂, C(O), O—C(O) or —NH-C(O), n is between 1 and 30, q is between 1 and 150, d3 and d4 correspond to 0 and/or 1, Q″ corresponds to —O-C(O) or —NH-C(O); and:
if X is a hydrogen atom, then p is equal to 0;
if X is a fluorine atom, then p is between 1 and 12;

and wherein the values a and c are integers, identical or different, greater than or equal to 1 and b is greater than or equal to 0; preferentially, a is between 1 and 10,000; b is between 1 and 5,000.

Preferably, the hydrocarbon-chain ASE polymers (ASE-H) consist of monomers of acrylic and/or methacrylic acid and C1-C4 alkyl acrylates.

Advantageously, the hydrocarbon-chain ASE polymers (ASE-H) comprise:
5 to 50% mol. acrylic and/or methacrylic acid;
50 to 95% mol. C1-C4 alkyl acrylates.

More preferentially, the ASE-H polymers comprise the following monomers:
10 to 20% mol. methacrylic acid (MA);
80 to 90% mol. ethyl acrylate (EA).

Preferably, the ASE-F non-associative polymers comply with the following general formula (II):

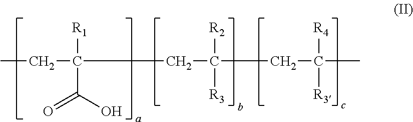

R1, R2 and R4 represent a hydrogen atom or a methyl group —CH₃;
R3 represents [Q]_{d1}-(CH₂)ₙ—H wherein n is between 1 and 30, d1 corresponds to 0 or 1, and Q corresponds to —C(O)—O or —C(O)—NH—;

or

R3 represents [Q]_{d2}-α, wherein:
d2 corresponds to 0 or 1;
Q corresponds to —C(O)—O or —C(O)—NH—; and
α corresponds to —C(CH₃)₃; —CH(CH₃)₂; —C(CH₃)₂—CH₂—C(CH₃)₃; —CN; —CH₂CH₂—N+(CH₃)₂(CH₂CO₂⁻); —CH₂CH₂—NH—C(CH₃)₃; —CH₂CH₂—N(CH₃)₂; pyrrolidinone; caprolactam;
R3′ represents [Q]_{d1}-(CH₂)ₙ—(CX₂)ₚX wherein n is between 1 and 30, d1 corresponds to 0 or 1, Q corresponds to —C(O)—O or —C(O)—NH—, X is a fluorine atom F and p is between 1 and 12;

and wherein the values a and c are integers, identical or different, greater than 1 and b is greater than or equal to 0; preferentially, a is between 1 and 10,000, b is between 1 and 5,000 and c is between 1 and 8,000.

These ASE-F polymers preferentially consist of monomers of:
methacrylic acid (MA),
2,2,2-trifluoroethyl methacrylate (TEEM) or trifluoroethyl acrylate or 2-perfluorobutylethyl acrylate or 2-perfluorohexylethyl acrylate or 2-perfluorooctylethyl acrylate; and
C1-C4 alkyl acrylate, preferentially ethyl acrylate (EA).

Introducing a fluorocarbon chain makes it possible, for cosmetic or pharmaceutical use, to reduce surface uptake of toxins.

Preferably, the fluorocarbon-chain ASE polymers (ASE-F) consist of the following monomers:
20 to 75% mol. acrylic and/or methacrylic acid;
0 to 30% mol. C1-C4 alkyl acrylates; and
10 to 55% mol. 2,2,2-trifluoroethyl methacrylate (TFEM) or trifluoroethyl acrylate or 2-perfluorobutylethyl acrylate or 2-perfluorohexylethyl acrylate or 2-perfluorooctylethyl acrylate.

More preferably, the fluorocarbon-chain ASE polymers (ASE-F) comprise monomers of methacrylic acid (MA), ethyl acrylate (EA) and 2,2,2-trifluoroethyl methacrylate (TEEM).

Advantageously, the fluorocarbon-chain ASE polymers (ASE-F) comprise:
50 to 60% mol. methacrylic acid (MA);
5 to 15% mol. ethyl acrylate (EA); and
30 to 40% mol. 2,2,2-trifluoroethyl methacrylate (TEEM).

Preferably, the hydrocarbon-chain HASE-H-RH polymers consist of monomers of methacrylic acid (MA), C1-C4 alkyl acrylates and a macromer which is an ester having the general formula (IV):

CH₂═CH(CH₃)—C(O)(OCH₂CH₂)_{q}OC(OC)(CH₂)ₙ—H (IV)
wherein q denotes a number between 5 and 10 and n is between 6 and 30 carbon atoms.

More preferably, the HASE-H-RH polymers comprise monomers of methacrylic acid (MA), ethyl acrylate (EA), and a macromer which an ester having a general formula (IV) as defined above, and thus comply with the following general formula (V):

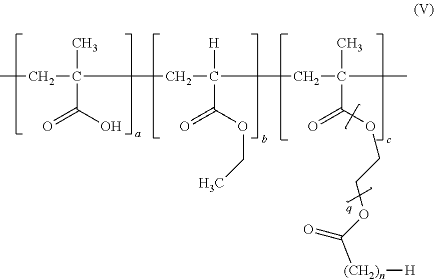

wherein:
q denotes a number between 5 and 10;
n is between 1 and 30;
a and c are integers, identical or different, greater than or equal to 1, and b is greater than or equal to 0; preferentially, a is between 1 and 10,000, b is between 1 and 10,000 and c is between 1 and 5,000.

More preferably, the HASE-H-RH polymers comply with general formula (V) and comprise:
5 to 85% mol. methacrylic acid (MA);
5 to 60% mol. ethyl acrylate (EA); and
1 to 90% mol. of a macromer which is an ester having general formula (IV) defined above.

The particularly preferred HASE-H-RH polymers comply with formula (V) above, and are such that:
q is equivalent to 7 and n is equal to 6 carbon atoms (hereinafter referred to as HASE-H-RH4 polymer);
q is equivalent to 7 and n is equal to 8 carbon atoms (hereinafter referred to as HASE-H-RH6 polymer);
q is equivalent to 9 and n is equal to 10 carbon atoms (hereinafter referred to as HASE-H-RH8 polymer).

Alternatively, according to the invention, the ethyl acrylate (EA) monomer of the HASE-H-RH polymer of formula (V) above may be replaced by a monomer of 2,2,2-trifluoroethyl methacrylate (TFEM) and thus corresponds to an HASE-F-RH polymer complying with the following general formula (VI);

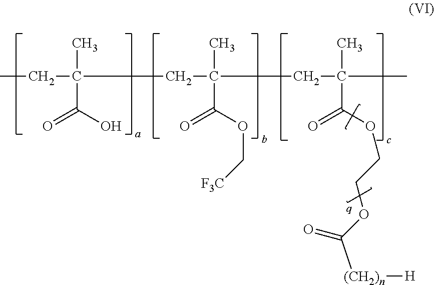

wherein:
q denotes a number between 5 and 10;
n is between 6 and 30 carbon atoms;
a and c are integers, identical or different, greater than or equal to 1, and b is greater than or equal to 0; preferentially, a is between 1 and 10,000, b is between 1 and 10,000 and c is between 1 and 5,000.

More preferably, the HASE-F-RH polymers comply with general formula (VI) above and comprise:
30 to 85% mol. methacrylic acid (MA);
0 to 50% mol. 2,2,2-trifluoroethyl methacrylate (TFEM); and
1 to 90% mol. of a macromer which is an ester having general formula (IV).

The particularly preferred HASE-F-RH polymers comply with formula (VI) above, and are such that:
q is equivalent to 7 and n is equal to 6 carbon atoms (hereinafter referred to as HASE-F-RH4 polymer);
q is equivalent to 7 and n is equal to 8 carbon atoms (hereinafter referred to as HASE-F-RH6 polymer);
q is equivalent to 9 and n is equal to 10 carbon atoms (hereinafter referred to as HASE-F-RH8 polymer).

Advantageously, the Applicant further succeeded in demonstrating that substituting hydrocarbon links with fluorocarbons on the macromer was possible in an HASE skeleton.

By modifying the skeleton thereof with fluorocarbon macromers in this way, the rheology-modifying polymer according to the invention can disperse the micro- or nanoparticles while providing the hydrophobicity and oleophobicity required for protection against chemical agents.

The structure of such fluorocarbon-chain HASE polymers (HASE-F), also suitable for use according to the invention, complies with the following general formula (VII):

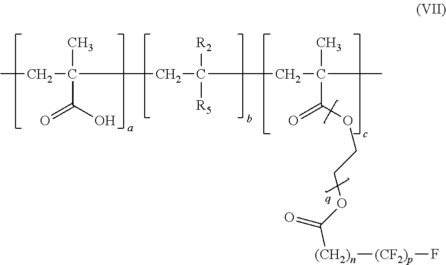

wherein:
R2 represents a hydrogen atom or a methyl group;
R5 represents [Q]_{d1}-(CH₂)ₙ—(CX₂)ₚX wherein n is between 1 and 30, d1 corresponds to 0 or 1, Q corresponds to —C(O)—O or —C(O)—NH—; and:
if X is a hydrogen atom, then p is equal to 0;
if X is a fluorine atom, then p is between 1 and 12;

or R5 represents [Q]_{d2}-α, wherein:
d2 corresponds to 0 or 1;
Q corresponds to —C(O)—O or —C(O)—NH—; and
α corresponds to —C(CH₃)₃; —CH(CH₃)₂; —C(CH₃)₂—CH₂—C(CH₃)₃; —CN; —CH₂CH₂—N+(CH₃)₂(CH₂CO₂⁻); —CH₂CH₂—NH—C(CH₃)₃; —CH₂CH₂—N(CH₃)₂; pyrrolidinone; caprolactam;
q denotes a number between 1 and 150;
n is an integer between 1 and 30;
p is an integer between 1 and 12;

and wherein the values a and c are integers, identical or different, greater than or equal to 1, and b is greater than or equal to 0; preferentially, a is between 1 and 10,000, b is between 1 and 10,000 and c is between 1 and 5,000.

More preferably, the HASE-F polymers comply with the following general formula (VIII):

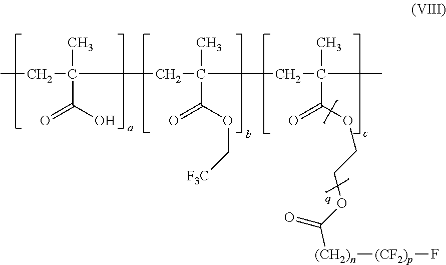

wherein:
q denotes a number between 5 and 10;
n is an integer between 1 and 30;
p is an integer between 1 and 12;
a and c are integers, identical or different, greater than or equal to 1, and b is greater than or equal to 0; preferentially, a is between 1 and 10,000, b is between 1 and 10,000 and c is between 1 and 5,000.

Even more preferentially, the HASE-F polymers comply with the general formula (VIII) above wherein:
q is equivalent to 5, 7 or 9;
n is equivalent to 2;
p is equivalent to 4, 6 or 8; and
a and c are integers, identical or different, greater than or equal to 1, and b is greater than or equal to 0; preferentially, a is between 1 and 10,000, b is between 1 and 10,000 and c is between 1 and 5,000. More preferably, the HASE-F polymers complying with general formula (VIII) comprise the following monomers:
5 to 85% mol. methacrylic acid (MA);
1 to 70% mol. 2,2,2-trifluoroethyl methacrylate (TFEM); and
1 to 50% mol. of a macromer which is an ester having general formula (IX):

CH₂═CH(CH₃)—C(O)(OCH₂CH₂)_{q}OC(O)(CH₂)₂—(CF₂)ₚF (IX)

wherein:
q is equivalent to 5, 7 or 9; and
p is equivalent to 4, 6 or 8.

The particularly preferred HASE-F polymers comply with formula (VIII) above, and are such that:
q is equivalent to 5 and p is equivalent to 4 (hereinafter referred to as HASE-F-RF4 polymer);
q is equivalent to 7 and p is equivalent to 6 (hereinafter referred to as HASE-F-RF6 polymer);
q is equivalent to 7 and p is equivalent to 6 (hereinafter referred to as HASE-F-RF8 polymer);

The HASE polymers described above may be synthesized using the same method as for ASE polymers. Details of a synthesis process of HASE-H and HASE-F polymers are given in example 5.

Particularly advantageously, the rheology-modifying or adapting polymers are chosen from the following polymers:
ASE-H;
ASE-F;
HASE-H-RH4;
HASE-H-RH6;
HASE-H-RH8;
HASE-F-RH4;
HASE-F-RH6;
HASE-F-RH8;
HASE-F-RF4;
HASE-F-RF6; or
HASE-F-RF8.

Even more preferentially, the rheology-modifying or adapting polymers are chosen from the following polymers:
HASE-H-RH4;
HASE-H-RH6;
HASE-H-RH8;
HASE-F-RH4;
HASE-F-RH6;
HASE-F-RH8;
HASE-F-RF4;
HASE-F-RF6; or
HASE-F-RF8.

Experiments made it possible to demonstrate that these polymers exhibit oleophobic rheological properties (viscoelasticity, flow, thixotropy, etc.) enabling the advantageous use thereof, as such:
in protection or decontamination of the skin (for example as a protective topical agent applicable to prevent contact with toxic chemicals),
in human medicine, notably in dermatology, for the prevention of skin irritations or allergies;
in cosmetics, notably as a protective epidermal barrier against external attacks.

### EXAMPLE 1

### Synthesis of 292 nm and 448 nm Silica Microparticles

The synthesis of the silica microparticles is carried out according to the Stöber method. This sol-gel method consists of hydrolyzing and condensing tetraethylorthosilicate (TEOS) in a mixture of alcohol, water and ammonia. The silica microparticle synthesis diagram using the Stöber method is illustrated in FIG. 2a.

The water/ammonia (H₂O/NH₃) ratio will control the particle size. Water will hydrolyze the particles whereas ammonia will stabilize them in the medium. The H₂O/NH₃ ratios used are 3.82 and 2.57 for 448 and 292 nm silica, respectively. The first reaction consists of hydrolyzing an —OEt group to —OH, and, in a second reaction, condensation will take place to form the silica microparticles.

Silicic acid is produced during hydrolysis and when the concentration thereof is above the solubility thereof in ethanol, it increases (nucleation) homogeneously and forms silica particles of submicronic size. This method is used for preparing pure silica particles.

Compounds may be incorporated by co-condensing TEOS with a silane (e.g. 3-aminopropyltriethoxysilane (APTES) which is a reactive amine containing silane) after the hydrolysis step. The particle diameter may be less than or equal to 10 nm and may range up to approximately 1 μm in diameter. They are generally non-uniform in size. Thus, further filtrations and separations are required to isolate the fractions of different sizes. The particle diameter may be controlled by means of the reaction temperature and the water/ammonia ratio in the reaction.

The procedure used makes it possible to produce silicas, of controlled sizes, functionalized with GPTMS (3-glycidyloxypropyltrimethoxysilane).

For this example, the silicas were functionalized with an amine group introduced using (3-aminopropyl)trimethoxysilane (AMS)), as illustrated in FIG. 2b.

Functionalization using for example the compounds ((aminomethyl)triethoxysilane, (aminomethyl)trimethoxysilane), ((5-aminopentyl)tri-ethoxysilane or (5-amino-pentyl)trimethoxysilane) could also be envisaged.

### EXAMPLE 2

### Functionalization of Silica Nanoparticles

Commercial 14 nm pyrogenated silica nanoparticles were functionalized with (3-aminopropyl)triethoxysilane in anhydrous toluene with a silica/aminosilane ratio of 10:1, as illustrated in FIG. 3.

Aminosilane will react by means of a condensation reaction with functionalized silica via the —OH groups.

For this experiment, it is important to work in an anhydrous medium so that the aminosilane is not hydrolyzed, which would induce condensation between the hydrolyzed Si—OH groups and nanoparticles of different sizes would be obtained.

After removing the toluene by centrifugation and washing with ethanol, the nanoparticles are stored in aqueous solution to prevent any contamination via the respiratory tract.

### EXAMPLE 3

### Synthesis of Titanium Nanoparticles

Titanium nanoparticles were obtained by functionalizing commercial nanoparticles in anatase form (due to the photocatalytic property thereof). Titanium dioxide nanoparticles may for example be functionalized with alkoxysilanes (formation of Ti—O—Si bonds) or by phosphorus compounds (formation of Ti—O—P bonds).

Phosphorus compounds, such as phosphonic acid or phosphonates for example, are known to form strong bonds with metal oxides and do not tend to be condensed like alkoxysilanes. To obtain titanium nanoparticles, the same functionalization method as that described above in example 2 for silica nanoparticles was used.

### EXAMPLE 4

### Synthesis of ASE Polymers

As illustrated in FIG. 4, the ASE polymers (ASE-H or ASE-F) may for example consist of methacrylic acid (MA), ethyl acrylate (EA) and/or 2,2,2-trifluoroethyl methacrylate (TEEM).

These polymers may be synthesized by polymerization in emulsion with sodium dodecyl sulfate (SDS) as a surfactant and acetone as a co-solvent. Two polymers were synthesized: ASE-H and ASE-F.

Synthesis of a polymer prior to grafting the micro- or nanoparticles makes it possible to change the polymer chain according to the application or the properties sought for the micro- or nanocomposite.

Introducing a fluorinated monomer has an effect on the stability of the emulsion during polymerization and an increase in the coagulation phenomenon was observed when the length and the quantity of fluorinated monomers increases, inducing a decrease in the conversion yields. In order to stabilize the system, 1% by mass of acetone is used as a co-solvent. This solvent is known to solubilize fluorinated monomers in emulsions. Yields of 88 and 51% for the ASE-H and ASE-F polymers were obtained after purification by dialysis (4000-6000 Da).

According to table 1 hereinafter, it can be seen that the quantity of methacrylic acid (MA) increases significantly when fluorinated monomer is added. This is probably due to the poorer integration of TEEM during the emulsion process. This may be confirmed by the decrease in the copolymer yield (51%).

TABLE 1
ASE-H and ASE-F polymer composition determined
by ¹H-NMR:

| | | | | | |
|---|---|---|---|---|---|
| | No. of | | | | |
| | protons | | Comp. | δH | δF |
| Resonance | (nominal) | Intensity | (% mol) | (ppm) | (ppm) |

ASE-H

| | | | | | |
|---|---|---|---|---|---|
| MA | 1 | 1000 | 17.8 | 12.3 | — |
| EA | 2 | 4620.6 | 82.2 | 4.01 | — |
| TFEM | 0 | 0 | 0 | — | — |

ASE-F

| | | | | | |
|---|---|---|---|---|---|
| MA | 1 | 1000 | 55.9 | 12.41 | — |
| EA | 2 | 179.8 | 10.0 | 3.96 | — |
| TFEM | 2 | 610.5 | 34.1 | — | 4.62 |

In table 1 above, MA relates to the resonance of the carboxylic acid proton, EA relates to the resonance assigned to the methylene proton of the ethyl ester and TFEM relates to the resonance of the methylene proton of the trifluoroethylene ester. The intensity of the resonance of MA is standardized at 1000 in each case and the intensities are standardized according to the nominal number of protons provided by the resonance.

### EXAMPLE 5

### Synthesis of HASE Polymers

As illustrated in FIG. 5, HASE polymers are synthesized using the same method as for ASE polymers. They consist of methacrylic acid (MA), ethyl acrylate (EA) and/or 2,2,2-trifluoroethyl methacrylate (TFEM) and of a hydrocarbon or fluorinated macromer.

According to FIG. 5, HASE-X-RXn will be the reference name of the synthesized polymer.

X corresponds to the letter H if the ethyl acrylate monomer is used. X corresponds to the letter F (HASE-F-RXn) if the fluorinated monomer is used.

RXn varies according to the type of chain, hydrocarbon (HASE-X-RHn) or fluorocarbon (HASE-X-RFn), and n varies according to the number of carbons in the macromer.

As the macromers (MHn or MFn) are not commercial products, they were synthesized by means of an esterification reaction of a polyethylene glycol monomethylmethacrylate with a hydrocarbon or fluorocarbon carboxylic acid compound, as illustrated in FIG. 6.

The reaction was catalyzed using a coupling agent, N,N′-dicyclohexylcarbodimide (DCC) in the presence of N,N′-dimethylpyridine (DMAP).

The synthesized compounds are obtained with different yields and are characterized by infrared or NMR. The synthesized compounds are listed in table 2 hereinafter. The number of PEG monomers, annotated m in FIG. 6, is determined by integrating the 1^{H}-NMR signals in the chemical shift ranges of 4.39-4.21 ppm (multiplet) and 3.76-3.64 ppm (multiplet). The initial polyethylene glycol monomethylmethacrylate being commercial and polydisperse, the chromatographic column made it possible to separate the different monomers. The yields of the monomers used in polymer synthesis are given in table 2.

TABLE 2
Summary of the chains used, macromer references
and reaction yields:

| | | Number m in | |
|---|---|---|---|
| | Macromer | macromer | |
| | reference | (determined | |
| Chain | (MKn) | by ¹H-NMR) | Yield (%) |
| C4H9 | MH4 | 7 | 11 |
| C6H13 | MH6 | 7 | 23 |
| C8H17 | MH8 | 9 | 24 |
| C4F9 | MF4 | 5 | 27 |
| C6F13 | MF6 | 7 | 63 |
| C8F17 | MF8 | 7 | 55 |

The HASE polymers were then synthesized according to the same polymerization method in emulsion as the ASE polymers. These polymers are thus synthesized by polymerization in emulsion with sodium dodecyl sulfate (SDS) as a surfactant and acetone as a co-solvent. Two polymers were synthesized: HASE-H and HASE-F. The composition of the polymerization medium is given in table 3. The macromer was introduced at a rate of 0.5% by mass.

TABLE 3
Mass composition of the polymers

| Compound (% mass) | HASE-X-RXn |
|---|---|
| MA | 8.25 |
| EA or TFEM | 10.9 |
| MXn | 0.5 |
| SDS | 1.42 |
| K₂S₂O₈ | 0.035 |
| Acetone | 1 |
| Water | 77.9 |

At the end of polymerization, the polymers obtained, in yields ranging from 52 to 81%, are purified by dialysis (4000-6000 Da) and characterized by IR, ¹H-NMR and 19F. The polymers synthesized and the molar composition calculated by ¹H-NMR are listed in table 4 hereinafter:

TABLE 4
Summary of the main polymers synthesized and the
molar composition thereof determined by ¹H-NMR:

| | | | |
|---|---|---|---|
| | No. of | | |
| | protons | | |
| Resonance | (nominal) | Intensity | Comp (% mol.) |

HASE-H-RH4

| | | | |
|---|---|---|---|
| MA | 1 | 1000 | 8.5 |
| EA | 2 | 1071.9 | 9.1 |
| MH4 | 24 | 9694.9 | 82.4 |

HASE-H-RH6

| | | | |
|---|---|---|---|
| MA | 1 | 1000 | 64.7 |
| EA | 2 | 492 | 31.9 |
| MH6 | 24 | 52.9 | 3.4 |

HASE-H-RH8

| | | | |
|---|---|---|---|
| MA | 1 | 1000 | 51.4 |
| EA | 2 | 921.4 | 47.3 |
| MH8 | 32 | 24.6 | 1.3 |

HASE-F-RH4

| | | | |
|---|---|---|---|
| MA | 1 | 1000 | 78.9 |
| TFEM | 2 | 0 | 0 |
| MH4 | 24 | 266.7 | 21.1 |

HASE-F-RH6

| | | | |
|---|---|---|---|
| MA | 1 | 1000 | 63.1 |
| TFEM | 2 | 482.5 | 30.5 |
| MH6 | 24 | 100.9 | 6.4 |

HASE-F-RH8

| | | | |
|---|---|---|---|
| MA | 1 | 1000 | 69.0 |
| TFEM | 2 | 400.1 | 27.6 |
| MH8 | 32 | 49.4 | 3.4 |

HASE-F-RF4

| | | | |
|---|---|---|---|
| MA | 1 | 1000 | 61.4 |
| TFEM | 2 | 41.6 | 2.6 |
| MF4 | 16 | 586.5 | 36.0 |

HASE-F-RF6

| | | | |
|---|---|---|---|
| MA | 1 | 1000 | 16.0 |
| TFEM | 2 | 4227.6 | 67.4 |
| MF6 | 24 | 1041.5 | 16.6 |

HASE-F-RF8

| | | | |
|---|---|---|---|
| MA | 1 | 1000 | 78.2 |
| TFEM | 2 | 236.8 | 18.5 |
| MF8 | 24 | 42.5 | 3.3 |

In table 4 above, MA relates to the resonance of the carboxylic acid proton, EA relates to the resonance assigned to the methylene proton of the ethyl ester, TFEM relates to the resonance of the methylene proton of the trifluoroethylene ester and MHn relates to the resonance of the protons of the ethoxylated units except for the four alpha protons of the two esters. The intensity of the resonance of MA is standardized at 1000 in each case and the intensities are standardized according to the nominal number of protons provided by the resonance.

Also, table 4 above suggests that the macromer MH4 is incorporated in the polymer to a greater degree than the others.

Comparing the different families of polymers, HASE-H-RHn, HASE-F-RHn and HASE-F-RFn, makes it possible to observe that, when the length of the hydrocarbon or fluorocarbon chain forming the macromer increases, the rate of incorporation of the macromer in the polymer decreases.

As regards the MA, EA and TFEM monomers, no linear variation of the rate of incorporation according to the type of macromers and the chain length is observed. DSC analysis made it possible to determine the vitreous transition temperature(s) of the polymers and the SEC analysis the mass of the polymers and the polydispersity index thereof. Two HASE polymers were characterized by SEC and the results are shown in table 5 hereinafter with the polymerization yields.

TABLE 5

| Polymer | Mw (g · mol−1) | Mw/Mn | Tg (° C.) | Yield (%) |
|---|---|---|---|---|
| HASE-H-RH8 | 1,162,632 | 4.48 | 54 | 64 |
| HASE-F-RF8 | 593,115 | 1.75 | 63 | 70 |

The difference in molar mass between the hydrocarbon and fluorocarbon polymers can be explained by the fact that introducing fluorinated chains induces the destabilization of the medium during the polymerization process, which creates shorter polymer chains.

### EXAMPLE 6

### Selecting the Preferred Polymer and Modifying Said Polymer with a View to Optimizing Same

A. Selecting the Preferred Polymer:

The various polymers synthesized underwent various analyses (rheological, dynamic light scattering, etc.) and goniometry analyses.

To carry out the goniometry analyses, polymer solutions were smeared on a model surface. 2.5 mg of each of the polymers was deposited on a glass plate over approximately 2-3 cm² and water was evaporated in the open air. Drops of olive oil were deposited on the surface, making it possible to determine the oleophilic/oleophobic properties of each polymer.

Three 3 μl drops of test liquid were deposited in order to calculate a mean. The results of this analysis are given in FIG. 7 illustrating the olive oil contact angles on polymers previously deposited on a glass plate.

Olive oil was chosen as this liquid has a surface tension similar to the toxic agents used for the study (γ=32 mN/m at 20° C.). Water was not used as, since the polymer is water-soluble, the film would be solubilized and the contact angle obtained would be non-significant.

As illustrated in FIG. 7, except for HASE-H-RH6, on the insertion of a monomer or a fluorocarbon chain, the value of the contact angle increases. This is observed particularly with HASE-F-RF8 polymer and its hydrocarbon counterpart which has a contact angle of 66° versus 25°, respectively. This value is also dependent on the length of the fluorocarbon chain as can be seen with an increase of 10 to 15° when the chain C₈F₁₇ is introduced compared to the chains C₄F₈ and C₆F₁₃. Also, compared to glass alone, a very small quantity of polymer makes it possible to increase the olive oil contact angle. It can thus be inferred that HASE-F-RF8 polymer has the best contact angle (Θ=66°) compared to other polymers.

In view of the different analyses carried out, by grouping rheological and goniometric studies, it was demonstrated that the most viscous polymer in the initial state is that having the best olive oil contact angle, i.e. HASE-F-RF8 polymer. For an application as a protective topical agent, the latter characteristic is very important as the toxin would not adhere, or would adhere very slightly, to the surface of the topical agent. As such, HASE-F-RF8 polymer was selected as the preferred polymer for the subsequent experiments. HASE-H-RH8 polymer was also studied in order to conduct certain analyses and for a comparison with the fluorocarbon counterpart thereof HASE-F-RF8.

b. Modification of the Preferred Polymer:

HASE-F-RF8 polymer with a molar macromer content of 3.3% having demonstrated advantageous surface (deposition) and rheological properties, the quantity of MF8 macromers was increased to increase the quantity of fluorinated chain in the medium. For this, two further polymers were synthesized: one containing 13.5% and the other containing 45.9% mol. of macromers. The synthesis process is polymerization in emulsion; the quantity of monomers introduced is given in table 6 hereinafter:

TABLE 6
Mass composition of the monomers introduced
during polymerization

| Compound (% mass) | HASE-F-RF8 (13.5%) | HASE-F-RF8 (45.9%) |
|---|---|---|
| MA | 6.75 | 8.25 |
| TFEM | 9.9 | 10.9 |
| MF8 | 3 | 0.5 |
| SDS | 1.42 | 1.42 |
| K₂S₂O₈ | 0.035 | 0.035 |
| Acetone | 1 | 1 |
| Water | 77.895 | 77.895 |

The molar composition of the monomers forming the polymers, obtained by ¹H-NMR, is given in table 7 hereinafter:

TABLE 7

| | | | |
|---|---|---|---|
| | No. of | | |
| Resonance | protons | Intensity | Comp (% mol.) |

HASE-F-RF8 (13.5%)

| | | | |
|---|---|---|---|
| MA | 1 | 1000 | 41.9 |
| TFEM | 2 | 1061 | 44.5 |
| MF8 | 24 | 322.5 | 13.5 |

HASE-F-RF8 (45.9%)

| | | | |
|---|---|---|---|
| MA | 1 | 1000 | 33.8 |
| TFEM | 2 | 600.6 | 20.3 |
| MF8 | 24 | 1361.8 | 45.9 |

The goniometry analyses conducted for these polymers HASE-F-RF8 (13.5% mol. macromers) and HASE-F-RF8 (45.9% mol. macromers), illustrated in FIG. 8, demonstrate that increasing the macromer content does not change the contact angle for 2.5 mg of polymers deposited. On the other hand, increasing the macromer content from 3.3% to 13.5% mol. increases the viscosity throughout the velocity gradient range, as illustrated in FIG. 9 presenting the flow curves for the polymers HASE-F-RF8 (3.3%), (13.5%) and (45.9%).

However, the behavior of the copolymer with a 45.9% mol. macromer content demonstrates that there is a threshold value from which the quantity of macromers no longer enhances the rheological properties.

### EXAMPLE 7

### Grafting of Silica Micro- or Nanoparticles with Polymers

Polymer/nanoparticle grafting was carried out using an amidation reaction under mild conditions.

The EDC/NHS ratio (in mole) used was [1:0.07]. The ratio of polymer/micro- or nanoparticles was calculated using the number of acid function equivalents borne by the polymer according to the number of amine function equivalents borne by the micro- or nanoparticles (for 1 acid function equivalent contained in the polymer, 1 amine function equivalent was introduced). At the end of the reaction, the reaction medium is purified by dialysis and centrifuged at 3300 rpm. The couplings produced in this study are summarized in table 8 hereinafter.

TABLE 8
Summary of polymer/silica couplings (1 silica equivalent):

| Polymer (1 eq) | Si-448 (1 eq) | Si-292 (1 eq) | Si-22 (1-eq) |
|---|---|---|---|
| ASE-H | X | X | X |
| ASE-F | X | X | X |
| HASE-H-RH8 | — | — | X |
| (1.3%) | | | |
| HASE-F-RF8 | — | X | X |
| (3.3%) | | | |

The polymers used for the grafts are ASE-H, ASE-F, HASE-H-RH8 (1.3% mol. macromers) and HASE-F-RF8 (3.3% mol. macromers).

The ASE-H and ASE-F polymers are used to construct the model and compare them to HASE polymers.

The polymer HASE-F-RF8 (3.3% mol. macromers) was chosen for the superior rheological and oleophobic properties thereof and using the carbon counterpart thereof makes it possible to compare the dispersion properties of the nanoparticles, according to the hydrocarbon or fluorocarbon chain, in an aqueous medium or on a substrate. The grafting compounds are annotated: ASE-H/Si-22 (1 eq) where ASE-H represents the polymer used, Si-22 the type of nanoparticle and the size and (1 eq) the number of amine functions in relation to the number of acid functions.

### EXAMPLE 8

### Analyses of Polymer/Silica (1 Equivalent) Compounds

Analyzing the dispersion of the micro- or nanoparticles following deposition on a substrate makes it possible to distinguish between certain compounds or nanoparticles according to the homogeneity or lack of homogeneity of the dispersion. For this, films of the compounds were prepared by depositing 100 μL of supernatant in a neutral medium on a glass plate and then the water was evaporated in a drier at atmospheric pressure. Based on the topographic images, featured in FIG. 10, compared to the size of the silica for the ASE-H and ASE-F compounds, as this decreases, the degree of aggregation decreases.

The depositions of compounds with ASE-F polymer exhibit few aggregations and the nanoparticles or particles are dispersed homogeneously unlike the compounds with ASE-H polymer which exhibit numerous aggregation zones.

The compound ASE-F/Si-22 is that having the deposition wherein the nanoparticles have the best dispersion.

In the light of the results detailed above, it would appear that the most preferred micro- or nanoparticles or the 22 nm silica nanoparticles because:
the quantity of polymer in the medium should advantageously be high to enable barrier protection, which is less the case for the 448 nm silica where few polymers are grafted thereon;
in order to obtain satisfactory protective efficacy from the nanoparticles, they should be dispersed homogeneously in the polymer. This was best observed for the 22 nm silica.

In addition, it is assumed that, due to the size thereof and the surface functionalization thereof, the quantity per cm² of 22 nm nanoparticles will be greater than the 448 and 292 nm particles. This enables for example greater uptake of the toxic agent by the active substance.

### EXAMPLE 9

### Introducing the Polymer/Nanoparticle Compounds into Cream and Gel Type Cosmetic Formulations

a. Formulation in a Cream:

The compounds were introduced into BariedermTech™ cream marketed by Uriage™.

The products were concentrated and then introduced into the cold cream at 10% by mass and at pH=9. The pH chosen is relatively high relative to the pH of the skin (pH≈5) and it is important not to harm it with a basic cream (risk of skin damage) but the compounds chosen have the best properties at around pH=9. The test compounds formulated in these creams were HASE-F-RF8 (3.3% mol. macromers)/Si (0.3 eq); HASE-F-RF8 (13.5% mol. macromers)/Si (0.3 eq).; HASE-F-RF8 (3.3% mol. macromers)/TiO2 (0.3 eq).

b. Formulation in a Gel:

Two types of gels were synthesized, a hydrophilic gel and a hydrophobic gel.

The gel formulations are detailed in table 9 hereinafter:

TABLE 9

| | |
|---|---|
| Hydrophilic gel | Hydrophobic gel |

| | Gel | | Gel |
|---|---|---|---|
| | (% | | (% |
| Compound | mass) | Compound | mass) |
| Water | 67.80 | HASE-F-RF8 (13.5%) | 1 |
| Carbopol ™ Ultrez 10 | 0.70 | Polymer/nanoparticles | 2.4 |
| EtOH (96%) | 30.00 | Water | 59.6 |
| TEA (99%) | 1.40 | EtOH (96%) | 25 |
| (50% in sol.) | | | |
| Polymer/nanoparticles | 0.10 | TEA (99%) (50% in | 2 |
| | | sol.) | |
| — | — | Fomblin ™ | 10 |

Fomblin™ is a perfluoropolyether (perfluorinated oil) which has two roles. The first is that of increasing the hydrophobicity and oleophobicity of the medium and the second is that of liquefying the gel. This second property was very useful as, at high polymer contents, the gel exhibits an elastic appearance and is not suitable for being applied correctly. The formula given in table 9 above is that suitable for obtaining a viscous gel with good application on the skin and a pH between 7.30 and 7.50.

### EXAMPLE 10

### Efficacy Test of Pure Product

The purpose of this experiment is that of determining the protective potential of a protective topical agent according to the invention by means of penetration kinetics on semi-permeable membranes, with respect to organophosphate compounds such as ethyl O-ethyl-O-(nitro-4-phenyl)phosphonate (paraoxon or PDX).

Tests on Synthetic Membrane:

The synthetic membrane used is a silicone (polydimethylsiloxane) membrane 400±100 μm thick marketed by Silicone Products (Nuneaton, UK).

The membrane is cut up into disks of approximately 10 cm2. The permeation test was conducted in glass Frantz type static diffusion cells (cells manufactured by a glass manufacturer: Laboratoire VERRE LABO-MULA, Corbas, France). The receiver medium is filled with “Hank's Balance Salt Solution” (HBSS).

TABLE 11
Breakdown of membranes relative to products.

| Products | Number of membranes |
|---|---|
| Control (untreated) | 3 |
| HASE-F-RF8 (13.5%) | 4 |
| HASE-F-RF8 (13.5%)/Si | 4 |

Treatment:

The protective topical agent according to the invention is applied at a rate of 5 mg/cm², spread using a flexible silicone spatula.

The membrane is then deposited onto the receiver compartment. A Teflon™ seal is added onto the membrane and the cell is sealed with the donor compartment, leaving a membrane exposure surface area of 1.13 cm². When the cell is sealed, it is placed on a cell-holder arranged in a water-bath, and a screw is added to enable satisfactory contact between the membrane and the receiver medium. Finally, the membrane and the gel are temperature-stabilized for 20 min. The water-bath is set to 38° C. so as to obtain a temperature of 32° C.±1° C. on the membrane surface.

The toxin is deposited at the center of the membrane in drop form. The quantity of paraoxon (PDX) deposited is 5 mg/cm², i.e. 4.9 μl.

400 μl samples are taken in the sampling loop every 1 hr30 min for PDX. Once the samples have been taken, an identical volume of HBSS is added in order to keep the quantity of receiver medium constant. All the samples are stored in a freezer at −20° C.

Assay of Quantity of PDX in the Receiver Media:

The method used is an enzymatic assay of the toxin. This assay method is an indirect method suitable for assaying the activity of an enzyme in the presence of paraoxon (PDX). The concentration of paraoxon in the sample is determined on the basis of a clearly defined concentration range and is proportional to the degree of inhibition of the enzyme (butyrylcholinesterase) added in known quantity in each sample.

The results are shown in FIG. 11 featuring the evaluation of the product with respect to transmembrane penetration of paraoxon. % Q0 represents the percentage absorbed dose of paraoxon.

It is observed that the transmembrane penetration of paraoxon is slowed down and reduced slightly when the membrane is pretreated with polymer HASE-F-RF8 (13.5% mol. macromers) but significantly with the compound HASE-F-RF8 (13.5% mol. macromers)/Si.

To compare the different products with each other, a non-parametric Kruskal-Wallis statistical test comparing the variances of more than two independent samples at t=6 h, followed by a Dunn test for comparing one sample with another, made it possible to draw conclusions on the protective effect of the compounds. The polymer does not have a significant protective effects; however, the polymer/silica compound has a significant protective effect with respect to the membrane.

This highlights the protective effect of the fluorocarbon polymer/micro- or nanoparticle compound synthesized on transmembrane PDX penetration.

### EXAMPLE 11

### Ecotoxicity of the Compounds According to the Invention

The aim was thus that of determining the ecotoxicological impact of the functionalized silica nanoparticles (448, 292 and 22 nm, annotated respectively in this study: 400, 300 and 22 nm) or pure nanoparticles (14 nm) and the grafting compound HASE-H-RH8 (1.3% mol. macromers)/Si (0.3eq).

Four test organisms were chosen from the sensitivity thereof, the representative nature of the ecosystem thereof and the trophic level (position occupied by an organism in the food chain: Chlorella vulgaris (SAPS) and Phaeodactylum tricornutum algae (fresh water and sea water), Daphnia Magna micro-crustacean (aquatic ecosystem) and Linum usitatissimum seeds (terrestrial ecosystem).

These tests were conducted under in vitro conditions in order to study the changes of the test organisms after incubation in contact with the chemicals and to determine the outcome of the chemicals after contact with the biological organisms. The results are represented schematically in FIGS. 12, 13 and 14.

FIG. 12 represents the EC₅₀ measurements for Daphnia magna of water suspensions.

FIG. 13 represents the EC₅₀ measurements of Phaeodactylum tricornutum and Chlorella vulgaris (SAPS) of water suspensions of the pure (SiO₂-14) and functionalized (SiO₂-22; -300 and -400) silica nanoparticles.

FIG. 14 represents the EC₅₀ measurements of Daphnia magna of water suspensions of the polymer HASE-H-RH8 (1.3% mol. macromers) (HASE polymer) and the compound HASE-H-RH8 (1.3% mol. macromers)/Si (0.3 eq) (grafted compound according to the invention).

These different toxicity tests with Daphnia magna, Phaeodactylum tricornutum and Chlorella vulgaris (SAPS) demonstrated that the toxicity of the nanoparticles decreases when the size of the compounds increases. These tests also demonstrate that the functionalization of 14 nm silica particles with the amine-silane compound (22 nm silica) induces a reduction in toxicity.

Similarly, the polymer alone and the grafted compound according to the invention exhibited a lower toxicity than the 22 nm nanoparticles.

The fourth test, with Linum usitatissimum, is a seed germination test suitable, by comparing the germination size (measurement method) relative to the control (a reference toxin: K2Cr2O7), for inferring the toxicity or not of the compound under study. The tests conducted did not show a difference in toxicity between the polymer alone (HASE-H-RH8 (1.3% mol. macromers) (HASE polymer)) and the grafted compound (HASE-H-RH8 (1.3% mo. macromers)/Si (0.3 eq)) according to the invention.

However, a notable difference between these two compounds (polymer alone and grafted compound) and the control was observed. The comparison of the 22 nm silica nanoparticles with the grafted compound demonstrated that the grafted compound according to the invention was significantly less toxic. For these experiments, the grafted compound is added at different concentrations to the seeds (0.01 mg/L, 0.1 mg/L, 1 mg/L, 10 mg/L and 100 mg/L), making it possible to determine the concentration from which the compound is toxic.

In view of the ecotoxicity experiments described above, it seems that the polymer HASE-H-RH8 (1.3% mol. macromers) is the least toxic of the compounds tested.

Furthermore, grafting nanoparticles with this polymer helps reduce the toxicity further, compared to 22 nm functionalized and 14 nm non-functionalized nanoparticles.

These results demonstrate that the compounds according to the invention have a reduced toxicity compared to the other products under test.

### EXAMPLE 12

### Example of Compounds According to the Invention

FIG. 15 represents a general diagram of compounds according to the invention suitable for obtaining new-technology protection. In the diagram in FIG. 15:
the groups R₁ correspond for example to hydrogen atoms or to alkyl groups having 1 to 4 carbon atoms;
the groups R₂ correspond to groups CₙX₂ₙ₊₁ where X represents a hydrogen or fluorine atom and n is between 1 and 9;
the groups R₃ correspond to a hydrocarbon or fluorocarbon chain CₙX₂ₙ₊₁ wherein X represents a hydrogen and fluorine atom and n is between 4 and 8;
p is between 5 and 10;
a, a′, a″, a′″ b, b′, b″, c, c′ and c″, identical or different, are integers, greater than 1; and
the balls or circles represent micro- or nanoparticles of aluminum oxide (Al₂O₂), copper oxide (CuO), iron oxide (Fe₂O₄ or γ-Fe₂O₃), magnesium oxide (MgO), silica (SiO₂), titanium dioxide (TiO₂) and zinc oxide (ZnO), having a nominal diameter of between 1 and 1500 nm.

### EXAMPLE 13

### Study of the Dispersion of Micro- or Nanoparticles in the Medium and Optimization of the Compounds According to the Invention

Dispersion of the nanoparticles being obtained in principle by means of the quantity of carboxylic acid functions present in the medium, the Applicant looked at reducing the number of silica equivalents. The quantity of grafted nanoparticles will decrease and the number of free carboxylic acid functions will increase in order to control the dispersion of the nanoparticles. The study on reducing the number of silica equivalents related to the polymer HASE-F-RF8 (3.3% mol. macromers) coupled with 22 nm silica at 0.8; 0.3; 0.1; 0.05 and 0.01 silica equivalent (number of amine function equivalents borne by the silica relative to the number of acid functions borne by the polymer) and the quantity of EDC was halved relative to the number of silica equivalents with a view to limiting the degree of grafting of acid functions but also in order to have free carboxylic acid functions in solution suitable for the dispersion of nanoparticles in a basic medium by means of ionic interactions. The EDC/NHS ratio used is [1:0.07].

The applicant then determined the number of equivalents for which silica is best dispersed and observed whether the wettability properties were different. Atomic Force Microscopy (AFM) and goniometry studies were thus carried out.

a. Topographies of the films in neutral media of the compounds HASE-F-RF8 (3.3%) Si-22 at 1; 0.3; 0.1; 0.05 and 0.01 equivalent:

Based on the topographic images illustrated in FIG. 16, it is observed that the deposition at 1 equivalent contains a high degree of aggregates unlike the four other depositions. In the case of the depositions of the formulations at 0.1 and 0.05 equivalent, the distribution of the nanoparticles therein is low. For the depositions of the formulations at 0.3 and 0.01 equivalent, satisfactory nanoparticle dispersion is observed in addition to low aggregation. These formulations would appear to the best equivalents for nanoparticle dispersion.

b. Goniometric Characterization:

As illustrated in FIG. 17, these five depositions were also characterized by means of goniometry (using water (for hydrophilicity/hydrophobicity) and olive oil (for oleophilicity/oleophobicity and having a surface tension similar to that of the chemical agent VX).

Based on FIG. 17, it can be inferred that, when the number of silica equivalents is reduced from 1 to 0.05 equivalent, the contact angle with water increases by 30° to approximately 70°, then decreases to 60° for 0.01 equivalent.

However, these values are not constant and, as shown by the histogram, at 0.1 and 0.05 equivalent, after one minute, the contact angle decreases to around 30° (value of the contact angle for 1 equivalent), which is explained by the solubilization of the polymer in aqueous medium. On the other hand, for the compound at 0.3 equivalent, the value of the angle (approximately 50°) remains constant even after one minute. The analysis with olive oil makes it possible to demonstrate that reducing the number of equivalents has no impact on the value of the contact angle, thus this value is around 25° for all the depositions. The contact angles with olive oil remain constant after one minute.

By comparing the topographic images obtained by AFM and the contact angle values obtained by goniometry, coupling with a number of nanoparticle equivalents of 0.3 equivalent demonstrated the best results in respect of dispersion and oleophobicity.

### EXAMPLE 14

### Example of Compounds According to the Invention

The Applicant tried in this example to optimize the efficacy of the product HASE-F-RF8 (13.5%) grafted with silica nanoparticles (HASE-F-RF8/Si) in a protective topical agent for use in protection or decontamination of the skin with respect to biological or chemical hazard agents.

Numerous studies have, in the past, created highly oleophobic surfaces with a contact angle between 90° and 140° with liquids having low surface tensions (γ), such as for example hexadecane (γ=27.3 mN/m) or olive oil (γ=32 mN/m), using perfluorinated compounds and structuring of the surface. The compound HASE-F-RF8/Si having also exhibited satisfactory dispersion of the silica nanoparticles in solution and following depositions on surfaces, it would be interest to increase this dispersion and the efficacy of protection.

HASE-F-RF8/Si further having free amine functions situated on the nanoparticles, it is possible to graft a molecule once again. For this, 4,4,5,5,6,6,7,7,7-nonafluoroheptanoic acid (RF4), 4,4,5,5,6,6,7,7,8,8,9,9,9-tridecafluorononanoic acid (RF6) and 4,4,5,5,6,6,7,7,8,8,9,9,10,10,11,11,11-heptadecafluorodecanoic acid (RF8) were bonded covalently with the compound HASE-F-RF8-Si, in aqueous medium and in the presence of a coupling agent, in order to increase the oleophobicity of the depositions. As illustrated in FIG. 18, the free amine functions borne by the silica nanoparticles react with the carboxylic acid functions of the fluorinated compounds RF4, RF6 and RF8 to obtain the compounds HASE-F-RF8/Si/RFn where n=4, 6 and 8.

This novel hybrid organic/inorganic compound HASE-F-RF8/Si/RF8 suspended in water was characterized by Dynamic Light Scattering (DLS). The wettability of the compounds HASE-F-RF8/Si and HASE-F-RF8/Si/RFn during depositions by means of smearing or spraying was also determined and the surface condition analyzed by Scanning Electron Microscopy (SEM) and Atomic Force Microscopy (AFM).

a. Grafting of Fluorinated Compounds on the Compound HASE-F-RF8/Si:

A [1:2] mixture of EDC/NHS (in mole) was added to a solution of 4,4,5,5,6,6,7,7,8,8,9,9,10,10,11,11,11-heptadecafluorodecanoic acid (RF8) or 4,4,5,5,6,6,7,7,8,8,9,9,9-tridecafluorononanoic acid (RF6) or 4,4,5,5,6,6,7,7,7-nonafluoroheptanoic acid (RF4) in water at a pH between 4 and 5. The reaction medium was stirred for 2 hours at 28° C. A solution at pH=8.5 of the compound HASE-F-RF8/Si (1 mole) was then added and the reaction continued for 5 days at 28° C. to obtain the compounds HASE-F-RF8/Si/RFn where n=4, 6 and 8. The compounds obtained were purified by dialysis (4000-6000 Da) and analyzed.

b. Analyses of the Compounds HASE-F-RF8/Si/RFn:

The reaction between the free amine functions borne by the nanoparticles and the carboxylic acid functions of the compounds RFn where n=4, 6 and 8 make it possible to obtain the compounds HASE-F-RF8/Si/RFn. The compounds RFn are previously activated with EDC and NHS in water to enable the intramolecular reaction between the carboxylic acid groups and the amine functions of the compound HASE-F-RF8/Si. Grafting induces the creation of an amide function (—CO—NH—). FIG. 19 represents the infrared spectrum of the compounds HASE-F-RF8/Si and HASE-F-RF8/Si/RFn. There is no difference between these graphs as the functions in these compounds are the same. In neutral medium, the carboxylic acid functions are partially ionized and the carbonyl band is shifted from 1708 cm⁻¹ to 1570 cm⁻¹ whereas the carbonyl band of amide remains at 1637 cm⁻¹ and the ester band at 1741 cm⁻¹. Furthermore, the C—F band of the fluorinated compounds, the C—O band of the ester and the Si—O—Si band are at 1285 cm⁻¹, 1245 cm⁻¹ and 1107 cm⁻¹, respectively. The infrared analysis showed the presence of free carboxylic acid functions which are necessary for the swelling of the compound in water and the dispersion of the nanoparticles in solution.

For the compounds HASE-F-RF8/Si and HASE-F-RF8/Si/RF8, the quantity of fluorine and carbon was estimated by elemental analysis.

The results of this elemental analysis of HASE-F-RF8/Si and HASE-F-RF8/Si/RF8 are included in table 12 hereinafter:

TABLE 12

| Compound | C (%) | N (%) | F (%) |
|---|---|---|---|
| HASE-F-RF8/Si | 29.65 | 0.69 | 9.20 |
| HASE-F- | 26.36 | 1.03 | 12.84 |
| RF8/Si/RF8 | | | |

The fluorine percentage value increases for HASE-F-RF8/Si/RF8 (F: 12.84%) unlike HASE-F-RF8/Si (F: 9.20%). This demonstrates the grafting between the carboxylic acid groups of the compound RF8 and the amine functions of the compound HASE-F-RF8/Si.

c. Depositions of Compounds:

The compounds HASE-F-RF8/Si and HASE-F-RF8/Si/RFn where n=4, 6 and 8 were deposited on 5 cm² glass plates by smearing and spraying. Smearing was performed by spreading the compounds on the plate with a spatula. Spray coating was performed with a compressed air spray gun having a 0.3/0.5 mm diameter nozzle and at a distance from the sample of 10 cm. 2%, 10.7% and 7.5% solutions were used for the compounds HASE-F-RF8/Si/RF4, HASE-F-RF8/Si/RF6 and HASE-F-RF8/Si/RF8, respectively.

d. Analysis of the Dispersion of the Nanoparticles in Solution

The dispersion of the nanoparticles in water is dependent on the pH of the suspension. In order to determine the pH of the suspension for which the best stabilization is obtained, the zeta potential of the compounds HASE-F-RF8, HASE-F-RF8/Si and HASE-F-RF8/Si/RF8 was measured in the pH range of 3 to 9. These measurements are included in FIG. 20. For HASE-F-RF8/Si at a pH of approximately 4, the zeta potential is approximately −20 mV due to the protonation of the ammonium groups to ammoniums unlike HASE-F-RF8/Si/RF8 which reaches a zeta potential of −30 mV.

This difference highlights the decrease of the amine groups in the compound HASE-F-RF8/Si/RF8 and justifies grafting the compound RF8 onto the silica nanoparticles. When the pH increases, a significant reduction is the zeta potential is measured and corresponds to the ionization of the carboxylic groups borne by the copolymer. At the same time, the ammonium groups start to be neutralized until they reach a zeta potential of approximately −70 mV. Maximum stabilization is obtained at pH=7 both for HASE-F-RF8/Si/RF8 and for HASE-F-RF8/Si. A stability study at pH=7 of HASE-F-RF8/Si/RF8 was conducted for 21 hours. The concentration of the solution used is 2% by mass.

The results demonstrate that:
the medium is stable for 4 hours and settling of the medium is observed from 4 hours;
after 21 hours, if the compound is stirred, it is re-dispersed and;
settling is once again observed after 4 hours.

e. Study of Olive Oil Wettability of the Compounds HASE-F-RF8/Si and HASE-F-RF8/Si/RFn.

The compounds HASE-F-RF8/Si and HASE-F-RF8/Si/RFn are deposited by smearing on glass plates, at a pH close to 7, varying the quantity from 0 to 60 mg. Furthermore, 15 mg of HASE-F-RF8/Si/RF8 is deposited using the spraying method and compared to the surface obtained with the smearing method.

In the case of HASE-F-RF8/Si/RF4 and HASE-F-RF8/Si/RF6, 1 mg and 1.5 mg respectively were deposited by spraying. The oleophobicity of the surfaces of the hybrid organic/inorganic compounds was evaluated by measuring the static contact angle of 4.9 μL drops of olive oil (γL=32 mN/m). In the case of HASE-F-RF8/Si and HASE-F-RF8/Si/RF8, the variation of the contact angle according to the quantity of compound (mg) is given in FIG. 21. Using the smearing method, oleophobic coatings are obtained rapidly from 5 mg of compound deposited with a contact angle of 90° for both compounds. When the quantity deposited onto the glass plate is increased, the contact angle for HASE-F-RF8/Si remains constant but the standard deviation increases. This deviation may be explained by the fracture of the film and the non-adhesion thereof on the glass substrate. The oil then spreads on the glass plate and on the HASE-F-RF8/Si coating. For HASE-F-RF8/Si/RF8, the contact angle increases continually according to the quantity deposited to obtain a high oleophobic surface with a contact angle of 120°. Grafting the fluorocarbon chain on HASE-F-RF8/Si increases the contact angle by 40° and makes it possible to switch from an oleophobic coating (HASE-F-RF8/Si) to a highly oleophobic coating (HASE-F-RF8/Si/RF8), as illustrated in FIGS. 22a and 22b. Furthermore, the adhesion of the compound HASE-F-RF8/Si is enhanced.

To obtain a superoleophobic surface, it is necessary to combine oleophobic compounds and micro- and nano-structuring of the surface. Probably, when the fluorocarbon compound RF8 was grafted, the structuring of the surface changed.

FIGS. 23a to 23f show scanning electron microscopy (SEM) analyses of the compounds HASE-F-RF8/Si and HASE-F-RF8/Si/RF8 using the smearing and spraying methods.

FIGS. 23a and 23b correspond to the deposition of 52 mg of HASE-F-RF8/Si with a smearing method.

FIGS. 23c and 23d correspond to the deposition of 47 mg of HASE-F-RF8/Si/RF8 with a smearing method.

FIGS. 23e and 23f correspond to the deposition of 15 mg of HASE-F-RF8/Si/RF8 with a spray method.

With the smearing method, HASE-F-RF8/Si has no surface structuring and the nanoparticles can be observed on the surface of the coating. On the other hand, the compound HASE-F-RF8/Si/RF8 exhibits micro- and nano-structuring due to the needle-shaped organization and the presence of nanoparticles but also oleophobicity provided by the fluorocarbon chains. All these results are the same for the spray coating of the compound HASE-F-RF8/Si/RF8.

As derived from FIGS. 24a and 24b, the spray deposition method does not impact the surface morphology and the contact angle which are identical using both methods: Θ=106°±5 for the smearing method (FIG. 24a) and Θ=111°±4 for the spraying method (FIG. 24b).

The needle-type structuring of the compound was also demonstrated by means of Atomic Force Microscopy (AFM) for both methods, as illustrated in FIGS. 25a and 25b.

For the compound HASE-F-RF8/Si/RF6, the measurement of the contact angle as a function of the quantity of compound smeared does not exhibit as great a variation as for the compound HASE-F-RF8/Si/RF8. Between 2 mg and 60 mg of compound deposited by smearing, the contact angle measured is 80.6°±3.1 to 88.6°±1.1, respectively. The deposition of the compound is not homogeneous. This is confirmed by the image obtained by Atomic Force Microscopy (AFM), of the deposition which exhibits a hole and furthermore no particular structure is observed, proving the low variation of the contact angle (see FIGS. 26a and 26b). With spraying, the contact angle obtained is 84.6°±1.9 (2 mg deposited) which is similar with the value obtained with the smearing method.

For the compound HASE-F-RF8/Si/RF4, the measurement of the contact angle as a function of the quantity of compound smeared does not exhibit as great a variation as for the compound HASE-F-RF8/Si/RF8, but above all the value of the angle is lower than for HASE-F-RF8/Si/RF6 and HASE-F-RF8/Si/RF8. Between 1.2 mg and 49 mg of compound deposited by smearing, the contact angle measured is 80.6°±1.8 to 76.6°±4.3, respectively. In this case, the contact angle decreases when the quantity deposited increases. It was observed that the adhesion of the substrate was not optimal when the quantity deposited increased but also that the deposition had cracks. This explains this decrease of the contact angle and the increase of the standard deviation. Accounting for the increase in the standard deviation, the value of the angle remains around 80° regardless of the quantity deposited. As for the compound HASE-F-RF8/Si/RF6, the Atomic Force Microscopy (AFM) deposition analysis (see FIGS. 26a and 26b) does not show any particular structuring.

FIG. 26a represents an AFM image of the smeared depositions of the compound HASE-F-RF8/Si/RF4 for 49 mg. FIG. 26b represents an AFM image of the smeared depositions of the compound HASE-F-RF8/Si/RF6 for 60 mg. With spraying, the contact angle is 88.1°±2.6 (1 mg deposited), which is slightly higher than the value obtained with the smearing method.

1. A compound comprising functionalized micro- or nanoparticles, covalently associated with rheology-modifying or -adapting polymers, wherein:
the functionalized micro- or nanoparticles are functionalized inorganic or metal oxide micro- or nanoparticles, the micro- or nanoparticles having a nominal diameter of between 1 and 1500 nm; and
the rheology-modifying or -adapting polymers are chosen from non-associative polymers or associative polymers.

2. The compound according to claim 1, wherein the micro- or nanoparticles are amine functionalized micro- or nanoparticles of inorganic or metal oxide.

3. The compound according to claim 2, wherein the amine functionalized micro- or nanoparticles are amine functionalized micro- or nanoparticles of silica (SiO₂) or titanium dioxide (TiO₂).

4. The compound according to claim 1, wherein the amine functionalized micro- or nanoparticles have an amine function content between 0.1 and 10 meq/g of micro- or nanoparticles.

5. The compound according to claim 3, wherein the amine functionalized micro- or nanoparticles of silica (SiO₂) or of titanium oxide (TiO₂) are nanoparticles, wherein the mean nominal diameter is between 5 and 500 nm and between 1 and 300 nm, respectively.

6. The compound according to claim 1, wherein the rheology-modifying or adapting polymers are selected from the group consisting of:
(i) alkali-swellable emulsion non-associative polymers comprising a hydrocarbon chain (ASE-H), the ASE-H non-associative polymers having the following general formula (I): 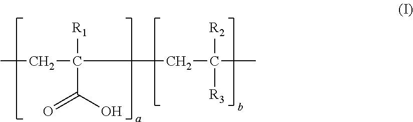
wherein:
R1 and R2 represent a hydrogen atom or a methyl group —CH₃;
R3 represents [Q]_{d1}-(CH₂)ₙ—H wherein n is between 1 and 30, d1 corresponds to 0 or 1, and Q corresponds to —C(O)—O or —C(O)—NH—;
or
R3 represents [Q]_{d2}-α, wherein:
d2 corresponds to 0 or 1;
Q corresponds to —C(O)—O or —C(O)—NH—; and
α corresponds to —C(CH₃)₃; —CH(CH₃)₂; —C(CH₃)₂—CH₂—C(CH₃)₃; —CN; —CH₂CH₂—N+(CH₃)₂(CH₂CO₂⁻); —CH₂CH₂—NH—C(CH₃)₃; —CH₂CH₂—N(CH₃)₂; pyrrolidinone; caprolactam;
and wherein the values a and b are integers, identical or different, greater than 1;
(ii) alkali-swellable emulsion non-associative polymers comprising a fluorocarbon chain (ASE-F), the ASE-F non-associate polymers having the following general formula (II): 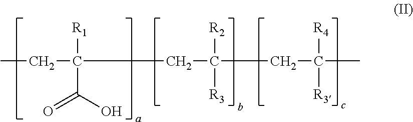
wherein:
R1, R2 and R4 represent a hydrogen atom or a methyl group —CH₃;
R3 represents [Q]_{d1}-(CH₂)ₙ—H wherein n is between 1 and 30, d1 corresponds to 0 or 1, and Q corresponds to —C(O)—O or —C(O)—NH—;
or
R3 represents [Q]_{d2}-α, wherein:
d2 corresponds to 0 or 1;
Q corresponds to —C(O)—O or —C(O)—NH—; and
α corresponds to —C(CH₃)₃; —CH(CH₃)₂; —C(CH₃)₂—CH₂—C(CH₃)₃; —CN; —CH₂CH₂—N+(CH₃)₂(CH₂CO₂⁻); —CH₂CH₂—NH—C(CH₃)₃; —CH₂CH₂—N(CH₃)₂; pyrrolidinone; caprolactam;
R3′ represents [Q]_{d1}-(CH₂)ₙ—(CX₂)ₚX wherein n is between 1 and 30, d1 corresponds to 0 or 1, Q corresponds to —C(O)—O or —C(O)—NH—, X is a fluorine atom F and p is between 1 and 12;
and wherein the values a and c are integers, identical or different, greater than 1 and b is greater than or equal to 0; and
(iii) macromeric hydrophobically modified alkali-swellable emulsion (HASE) associative polymers having a hydrocarbon chain (HASE-H-RH or HASE-F-RH) or a fluorocarbon chain (HASE-F-RF) complying with the following general formula (III): 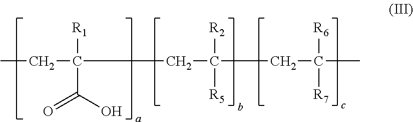
wherein:
R1, R2 and R6 represent a hydrogen atom or a methyl group;
R5 represents [Q]_{d1}-(CH₂)ₙ—(CX₂)ₚX wherein n is between 1 and 30, d1 corresponds to 0 or 1, Q corresponds to —C(O)—O or —C(O)—NH—; and:
if X is a hydrogen atom, then p is equal to 0;
if X is a fluorine atom, then p is between 1 and 12;
or
R5 represents [Q]d2-α, wherein:
d2 corresponds to 0 or 1;
Q corresponds to —C(O)—O or —C(O)—NH—; and
α corresponds to —C(CH₃)₃; —CH(CH₃)₂; —C(CH₃)₂—CH₂—C(CH₃)₃; —CN; —CH₂CH₂—N+(CH₃)₂(CH₂CO₂⁻); —CH₂CH₂—NH—C(CH₃)₃; —CH₂CH₂—N(CH₃)₂; pyrrolidinone; caprolactam;
R7 represents [Q′]_{d3}-(OCH₂CH₂)_{q}-[Q]_{d4}-(CH₂)ₙ(CX₂)ₚX wherein Q′ corresponds to —CH₂, C(O), O—C(O) or —NH—C(O), n is between 1 and 30, q is between 1 and 150, d3 and d4 correspond to 0 and/or 1, Q″ corresponds to —O—C(O) or —NH—C(O); and:
if X is a hydrogen atom, then p is equal to 0;
if X is a fluorine atom, then p is between 1 and 12;
and wherein the values a and c are integers, identical or different, greater than or equal to 1 and b is greater than or equal to 0.

7. The compound according to claim 6, wherein the rheology-modifying or adapting polymers are selected from the group consisting of:
ASE-H;
ASE-F;
HASE-H-RH4;
HASE-H-RH6;
HASE-H-RH8;
HASE-F-RH4;
HASE-F-RH6;
HASE-F-RH8;
HASE-F-RF4;
HASE-F-RF6; and
HASE-F-RF8.

8. The compound according to claim 6, wherein the rheology-modifying or -adapting polymers are associative polymers, and wherein a molar percentage of macromonomers or macromers is 3 and 50% of the polymer.

9. A protective topical agent comprising a compound according to claim 1, in a pharmaceutically and/or cosmetically acceptable medium.

10. A topical agent according to claim 9, further comprising at least one of (i) one or a plurality of detoxifying agents and (ii) one or a plurality of additional polymers.

11. Compound A medicinal product comprising the compound according to claim 1.

12. A method of preventing skin irritations or allergies, comprising administering the compound according to claim 1.

13. A method of protecting or decontaminating skin due to biological or chemical hazard agents, comprising administering the compound according to claim 1.

14. A method of protecting skin against toxic agents of organophosphate compound (OPC) family or against vesicant agents, comprising administering the compound according to claim 1.

15. A method for synthesizing a compound according to claim 1, comprising steps for:
mixing a coupling agent with a catalyst;
adding the mixture obtained to a solution of rheology-modifying or adapting polymers chosen from non-associative or associative polymers, in water;
stirring the reaction mixture;
adding functionalized micro- or nanoparticles of aluminum oxide (Al₂O₃), magnesium oxide (MgO), silica (SiO₂), titanium dioxide (TiO₂), zinc oxide (ZnO) or copper oxide (CuO) the micro- or nanoparticles having a nominal diameter between 5 and 1500 nm, and having been previously dispersed in aqueous phase in the reaction mixture;
purifying the reaction mixture by dialysis; and
retrieving the compound comprising one or a plurality of amine functionalized micro- or nanoparticles covalently associated with one or a plurality of rheology-modifying polymers.

16. The method according to claim 15, wherein the coupling agent is N-(3-dimethylaminopropyl)-N′-ethylcarbodiimide hydrochloride (EDC), and the catalyst is N-hydroxysuccinimide (NHS).

17. A polymer selected from the group consisting of:
(i) compounds complying with the following general formula (I): 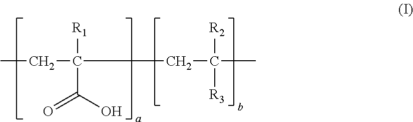
wherein:
R1 and R2 represent a hydrogen atom or a methyl group —CH₃;
R3 represents [Q]_{d1}-(CH₂)ₙ—H wherein n is between 1 and 30, d1 corresponds to 0 or 1, and Q corresponds to —C(O)—O or —C(O)—NH—;
or
R3 represents [Q]_{d2}-α, wherein:
d2 corresponds to 0 or 1;
Q corresponds to —C(O)—O or —C(O)—NH—; and
α corresponds to —C(CH₃)₃; —CH(CH₃)₂; —C(CH₃)₂—CH₂—C(CH₃)₃; —CN; —CH₂CH₂—N+(CH₃)₂(CH₂CO₂⁻); —CH₂CH₂—NH—C(CH₃)₃; —CH₂CH₂—N(CH₃)₂; pyrrolidinone; caprolactam;
and wherein the values a and b are integers, identical or different, greater than 1;
(ii) compounds complying with the following formula (II): 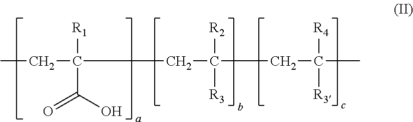
wherein:
R1, R2 and R4 represent a hydrogen atom or a methyl group —CH₃;
R3 represents [Q]_{d1}-(CH₂)ₙ—H wherein n is between 1 and 30, d1 corresponds to 0 or 1,
and Q corresponds to —C(O)—O or —C(O)—NH—;
or
R3 represents [Q]_{d2}-α, wherein:
d2 corresponds to 0 or 1;
Q corresponds to —C(O)—O or —C(O)—NH—; and
α corresponds to —C(CH₃)₃; —CH(CH₃)₂; —C(CH₃)₂—CH₂—C(CH₃)₃; —CN; —CH₂CH₂—N+(CH₃)₂(CH₂CO₂⁻); —CH₂CH₂—NH—C(CH₃)₃; —CH₂CH₂—N(CH₃)₂; pyrrolidinone; caprolactam;
R3′ represents [Q]_{d1}-(CH₂)ₙ—(CX₂)ₚX wherein n is between 1 and 30, d1 corresponds to 0 or 1, Q corresponds to —C(O)—O or —C(O)—NH—, X is a fluorine atom F and p is between 1 and 12;
and wherein the values a and c are integers, identical or different, greater than 1 and b is greater than or equal to 0; and
(iii) compounds complying with the following formula (III): 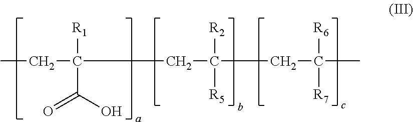
wherein:
R1, R2 and R6 represent a hydrogen atom or a methyl group;
R5 represents [Q]_{d1}-(CH₂)ₙ—(CX₂)ₚX wherein n is between 1 and 30, d1 corresponds to 0 or 1, Q corresponds to —C(O)—O or —C(O)—NH—; and:
if X is a hydrogen atom, then p is equal to 0;
if X is a fluorine atom, then p is between 1 and 12;
or
R5 represents [Q]_{d2}-α, wherein:
d2 corresponds to 0 or 1;
Q corresponds to —C(O)—O or —C(O)—NH—; and
α corresponds to —C(CH₃)₃; —CH(CH₃)₂; —C(CH₃)₂—CH₂—C(CH₃)₃; —CN; —CH₂CH₂—N+(CH₃)₂(CH₂CO₂⁻); —CH₂CH₂—NH—C(CH₃)₃; —CH₂CH₂—N(CH₃)₂; pyrrolidinone; caprolactam;
R7 represents -[Q′]_{d3}-(OCH₂CH₂)_{q}-[Q′]_{d4}-(CH₂)ₙ(CX₂)ₚX wherein Q′ corresponds to —CH₂, C(O), O—C(O) or —NH—C(O), n is between 1 and 30, q is between 1 and 150, d3 and d4 correspond to 0 and/or 1, Q″ corresponds to —O—C(O) or —NH—C(O); and:
if X is a hydrogen atom, then p is equal to 0;
if X is a fluorine atom, then p is between 1 and 12;
and wherein the values a and c are integers, identical or different, greater than or equal to 1 and b is greater than or equal to 0.

18. The compound according to claim 1, wherein the functionalized inorganic or metal oxide micro- or nanoparticles are selected from the group consisting of aluminum oxide (Al₂O₃), copper oxide (CuO), iron oxide (Fe₃O₄ or γ-Fe₂O₃), magnesium oxide (MgO), silica (SiO₂), titanium dioxide (TiO₂) and zinc oxide (ZnO).

19. The compound according to claim 2, wherein the micro- or nanoparticles are amine functionalized micro- or nanoparticles of inorganic or metal oxide selected from the group consisting of aluminum oxide (Al₂O₃), copper oxide (CuO), iron oxide (Fe₃O₄ or γ-Fe₂O₃), magnesium oxide (MgO), silica (SiO₂), titanium dioxide (TiO₂) and zinc oxide (ZnO).

20. The compound according to claim 4, wherein the amine functionalized micro- or nanoparticles of silica (SiO₂) or of titanium oxide (TiO₂) are nanoparticles, wherein the mean nominal diameter is between 5 and 500 nm and between 1 and 300 nm, respectively.
